(19) United States
(12) Patent Application Publication
(10) Pub. No.: US 2018/0214028 A1
(43) Pub. Date: Aug. 2, 2018

(54) SYSTEM FOR BODY TEMPERATURE MEASUREMENT

(71) Applicant: YONO HEALTH INC., Sunnyvale, CA (US)

(72) Inventors: Yunlong ZHANG, Sunnyvale, CA (US); Peng PENG, Sunnyvale, CA (US); Zehui XI, Sunnyvale, CA (US)

(73) Assignee: Yono Health Inc., Sunnyvale, CA (US)

(21) Appl. No.: 15/747,002

(22) PCT Filed: Jul. 25, 2016
(86) PCT No.: PCT/US2016/043870

§ 371 Date: Jan. 23, 2018

Related U.S. Application Data
(60) Provisional application No. 62/298,199, filed on Feb. 22, 2016.
(60) Provisional application No. 62/196,286, filed on Jul. 23, 2015.

(51) Int. Cl. A61B 5/01; A61B 5/00; A61B 10/00

(52) U.S. Cl. CPC A61B 5/01; A61B 5/4318; A61B 5/6817; G01K 13/002; A61B 2010/0019; A61B 5/4815; A61B 5/0008; A61B 10/0012

(57) ABSTRACT

The methods and apparatuses (including systems and device) described herein may be used to accurately and comfortably monitor and track body temperature using a wearable device that can be positioned inside a patient's ear for measuring the patient's temperature, sleeping quality, or other vital signs. These systems and methods may also include a base station and a remote analytics unit (e.g., software, hardware or firmware, including application software that may run on a hand-held or wearable electronics device). The inter-aural device (earplug device) may record and pre-process information that may then be adaptively passed on the remote analytics unit for further processing. The earplug device may engage the remote analytics unit with to download and process the temperature measurement data from the device, to electrically charge the device and also to communicate wirelessly with a smart phone or different types of mobile devices.

### CROSS REFERENCE TO RELATED APPLICATIONS

This patent application claims priority to U.S. provisional patent application no. 62/196,286 (“SYSTEM FOR TEMPERATURE MEASUREMENT”), filed on Jul. 23, 2015 and U.S. provisional patent application No. 62/298,199 (“SMART REAL-TIME HEALTH MONITORING HEADSET/EAR PLUG”), filed on Feb. 22, 2016, each of which is herein incorporated by reference in its entirety.

This patent application may be related to U.S. patent application Ser. No. 14/953,301 (“TEMPERATURE MEASURING DEVICE”), filed on Nov. 28, 2015, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are systems and method for monitoring body temperature using an earpiece that may be comfortably worn for an extended period of time to automatically and regularly (e.g., once every x minutes, where x is between 0.1 and 120) sample body temperature. In particular, described herein are systems and methods for comfortably monitoring body temperature while sleeping, and may include methods for predicting ovulation, measuring sleep quality, or other suitable activity or purpose.

### BACKGROUND

Body temperature may be used as a parameter in a wide variety of useful applications, including diagnosis of acute and chronic disorders, monitoring of body activity (including hormonal activity), and for therapeutic monitoring when applying heat/cold therapies. In particular, basal body temperature may be useful.

Basal body temperature typically refers to the lowest body temperature attained during rest (e.g., during sleep). Crude estimates of basal body temp (BBT) may be estimated by a temperature measurement immediately after awakening and before any physical activity has been undertaken. For more accurate results, it has been recommended to wear internally worn temperature loggers, particularly during sleep. Unfortunately such temperature loggers are often difficult to operate and uncomfortable.

In women, ovulation causes an increase of one-half to one degree Fahrenheit (one-quarter to one-half degree Celsius) in BBT; monitoring of BBTs is one way of estimating the day of ovulation. The tendency of a woman to have lower temperatures before ovulation, and higher temperatures afterwards, is known as a biphasic pattern. Charting of this pattern may be used as a component of fertility awareness. The BBT of men is comparable to the BBT of women in their follicular phase. Thus, measuring basal body temperature has been recognized as a way of determining a woman's time of ovulation during her fertility cycle. This may be useful for both avoiding pregnancy and for conceiving. Although BBT may be useful in detecting ovulation, it may also be difficult to distinguish. At the time of ovulation, a woman only experiences an increase of basal body temperature of about a quarter to 0.3° C. (about one-half degree Fahrenheit). Unless measured right around the temperature minimum, this slight increase may not be detected due to larger variations in the ambient background temperature.

To eliminate inconsistent basal body temperature readings, a woman typically measures her temperature daily at the same time and under the same conditions. However, even compliance to a strict schedule does not assure an accurate reading of the basal body temperature. This is because physiological events in the woman's body do not necessarily coincide with the time of when she goes to bed or wakes up. In addition, a single temperature measurement per day might not yield enough information to accurately determine the time of ovulation, since other causes, e.g., temporary insomnia, might be the origin of a sudden increase in temperature. Measuring a woman's other physiological functions, e.g., pulse and heart rate, in combination with data about her temperature during resting time, can improve the accuracy in predicting her ovulation time.

During sleep, the human body goes through several sleep cycles, where in each cycle an individual goes through different consciousness levels which are categorized as rapid eye movement (REM) and non-rapid eye movement (NREM). The REM type of sleep is associated with the capability of dreaming, while in the NREM type of sleep there is relatively very little dreaming. The sequence in a typical sleep cycle consists of different stages in the NREM sleep (Stages N1-N3, as categorized by the American Academy of Sleep Quality) followed by REM sleep. Published studies have shown that the number and duration of these NREM-REM cycles varies, with an average of 4 cycles for 8 hours of sleep. The average duration of each of these cycles varies with approximately 70-100 minutes for the first cycle, and 90-120 minutes for the second and later cycles. Individuals who suffer from sleep disorders (e.g., insomnia, sleep apnea, restless legs syndrome, narcolepsy) typically do not experience these sleep cycles fully, and thus could be prone to various health problems. It has also been established by studies that the timing of waking up is also important. As stage N3 in NREM sleep, and REM sleep, are the deepest sleep stages, a person who is awaken during these stages would often carry a feeling of drowsiness during the day. Thus, a feeling of better sleep quality could be obtained by timing the awakening of a person to be at the shallower stages of the sleep, e.g., NREM N1 and N2.

As the body goes through the NREM-REM sleep cycles, the body temperature is also expected to oscillate with very small amplitude of several tenths of degrees Celsius or less. As these cycles relate to the body temperature fluctuations, the number of temperature fluctuation cycles, their duration, and their amplitude, could provide valuable information about the sleep cycle. Thus, it would be useful to provide monitoring of body temperature (even without measuring BBT) during sleep.

Traditional thermometers are not well-suited for continuously measuring a person's temperature over an extended resting period. These thermometers lack the capability to measure other physiological functions. In addition, these thermometers are often invasive, e.g. rectal probe, require sterilization, are inconvenient to operate for longer periods, and are limited in their accuracy that is not high enough for determining an increase in temperature when a woman ovulates. For example, a thermometer measuring a patient's skin temperature often has low accuracy, since ambient temperature can readily alter its readings. Furthermore, traditional thermometers are not equipped to continuously record temperature data and analyze this data in real-time. For this reason, BBT readings are generally still recorded by hand and later inputted into computer software for further analysis to predict ovulation. Besides the inconvenience factor, this procedure increases the risk of introducing errors into the prediction by, e.g., inputting an incorrect value into the program. An erroneous reading for just one day may yield unusable results for the entire cycle. Since even slight errors in measuring the basal body temperature would result in a wrong prediction, it is critical that the temperature measurements minimize the error every day.

Although automatic, and wearable thermometers have been proposed (including those to be worn in the ear), such devices typically have not proven useful. User compliance is a major issue, particularly where the devices are worn while sleeping. In addition, continuous and accurate monitoring has proven challenging, as the battery life (particularly for wireless devices) is a concern. Thus, there is a need for an easy, accurate, and comfortable way of providing continuous temperature monitoring.

### SUMMARY OF THE DISCLOSURE

Described herein are apparatuses (e.g., devices, systems, etc.) and methods for monitoring body temperature, including but not limited to, detection of basal body temperature. These apparatuses and methods may include one or more of: an earpiece including a temperature sensor (e.g. thermistor), a base station for charging and storing the earpiece, and software, hardware, firmware, etc. (including a non-transitory computer-readable storage medium storing a set of instructions capable of being executed by a processor). The earpiece may be referred to as an ear implant, ear insert, earplug, or the like. The earpiece typically includes one or more temperature sensor, such as a resistive temperature detectors (e.g., thermistor, NTC/PTC, etc.), thermocouple, thermopile, IR thermosensors, etc. Additional sensors (e.g., electrical sensors, optical sensors, etc.) may also be included for determining other non-temperature parameters such as heart rate, blood pressure, pulse oxygenation, body movement, etc.

The earpiece may include an insertion shaft portion that projects into the ear canal, and an external seating body that seats in the outer ear (e.g., the pinna, and particularly the concha region). The insertion shaft typically extends distally from the external seating body. The insertion shaft may extend less than a 1.5 cm (e.g., less than 1.3 cm, less than 1.2 cm, less than 1.1 cm, less than 1.0 cm, less than 0.9 cm, less than 0.8 cm, less than 0.7 cm, less than 0.6 cm, less than 0.5 cm, etc.). The earpiece typically includes control circuitry, which may include one or more of a microcontroller, a memory, an analog-to-digital converter (ADC), power regulator circuits, and wireless communication circuitry (e.g., WiFi, Bluetooth, ZigBee, near field communication (NFC), etc.) including any associated antenna, and clock (e.g., timer) circuitry. Any of these components may be combined or integrated together (e.g., the microcontroller may include memory, wireless communication, timer(s), ADC, etc.). The microcontroller may be configured to operate in a low-energy mode, with the majority of the components, including the temperature sensor in a sleep or standby mode which uses very little power, during operation, and automatically and periodically transition to an active (wake) mode in which power is applied to the temperature sensor to detect temperature and/or other sensor data, process the measured data (e.g., average, filter, etc.), convert it to digital data and store the digital data in the memory. Any of these features may be optimized as described herein to improve accuracy, reduce the footprint and reduce the energy requirements. For example, the earpiece device may be configured to operate in a user (active) mode to take a sample only every 2-10 minutes or more (e.g., every 2 minutes, every 3 minutes, every 4 minutes, every 5 minutes, every 6 minutes, every 7 minutes, every 8 minutes, every 9 minutes, every 10 minutes, every 15 minutes, every 20 minutes, every 30 minutes, every 40 minutes, every 45 minutes, every 50 minutes, every 60 minutes, every 90 minutes, every 120 minutes, etc.). Samples may be taken and stored without a time/date stamp; e.g., the earpiece does not have to take a time/date data with the sample, as this information may be accurately determined upon docking and prior to transfer of the temperature data.

The temperature sensor in the earpiece may be thermally isolated from the external seating body by included an additional insulator (thermal barrier, insulative barrier, etc.) in the external seating body or between the external seating body and the insertion shaft, or at a portion of the insertion shaft proximal to the temperature sensor. The additional insulation has been found to increase the accuracy, particularly when these devices are worn in an active (e.g., non-sleeping) individual; airflow (e.g., wind) across the outer external seating body region may otherwise impact the accuracy of the temperature sensor, particularly when using a resistive temperature sensor such as a thermistor.

The earpiece, and particularly the microcontroller, may control the on-board (on the earpiece) activity and operation, but it may also control the operation of the base station. A base station typically includes a docking cradle for connection to the earpiece. The base until may include a battery and a power connector (e.g., to connect to wall power or other external power supply); thus, the base station may charge the earpiece when plugged in (e.g., to a USB port or USB charger) or when unplugged, by charging off of the base station battery. The base station may include a plurality of connections for connecting directly to the earpiece (e.g., pogo pin connectors, etc.) for charging and/or passing data. Alternatively or additionally, the base station may wirelessly communicate and/or charge the earpiece, such as by inductive charging/communication (e.g., in earpieces including an inductive charging antenna/coil).

Typically, the earpiece may transmit (e.g., to separate processor, such as a handheld or wearable devices including a smartphone, tablet or computer, and/or to a remote server) the data collected (sensor data). Transmission may occur only when the earpiece is in a docket mode, e.g., when the earpiece detects that it is docked and/or charging in the base station, which may save power and prevent loss of data fidelity. The earpiece may confirm that a reliable contact has been made between the base station and the earpiece before transmitting and/or charging. Because of the small size and position of the contacts on the earpieces (e.g., the connectors on the earpiece may be small, flush or recessed regions on an underside of the external seating body), the base station may be configured to establish and hold the connection by holding the earpiece in a particular orientation and by applying force to maintain the integrity of the electrical connection between the electrical contacts on the earpiece and the connectors on the base station. For example the base station may include a lid that is latched down and applies force to hold the earpiece in the docking cradle. The lid and/or earpiece may also be adapted to assist in holding the earpiece in the cradle; for example the lid may include a projection that mates with a depression on the external seating body to apply force appropriate to hold the contacts of the earpiece against the connectors in the base station. Further, the microcontroller may be configured to wait a predetermined wait time once contact with the base station is detected before transmitting data and/or charging. For example, the microcontroller may be configured to wait 2 or more seconds, 3 or more seconds, 4 or more seconds, 5 or more seconds, 10 or more seconds, 15 or more seconds, 20 or more seconds, 30 or more seconds, 45 or more seconds, 1 or more minutes, 2 or more minutes, 3 or more minutes, 4 or more minutes, 5 or more minutes, 6 or more minutes, 10 or more minutes, etc. The earpiece may be configured to allow charging during this time period.

As mentioned, any of the apparatuses (e.g., systems) described herein may also include software, firmware, and/or hardware for receiving, analyzing, presenting and transmitting data or materials derived from the data (e.g., temperature data) collected by the earpiece. For example, the apparatus may be adapted to transmit the data to a smartphone running an app (application software) that receives the data, determine the time/date information corresponding to the temperature data, and calculates basal body temperature so that it may be displayed, or transmits it to a remote server for calculation. This data may be graphically displayed. The smartphone may also collect user information that may be combined with the temperature information. This application software may operate as a log, recording in a calendar the temperature information. The apparatus may also calculate (or transmit to a remote site to calculate, then later receive) ovulation or fertility predictive information or other therapeutic or diagnostic information.

In general, the apparatuses described herein are configured for long-term (e.g., many hours, e.g., greater than 4, greater than 5, greater than 6, greater than 8, greater than 10, greater than 12, etc.), comfortable wear, as well as for long-term operation without requiring a charge. In general, the shape of the earpiece (e.g., the insertion shaft and the external seating body) may be configured to be comfortably worn. For example, the insertion shaft may be small, and may include an sealing member that is configured to seal the distal end of the insertion shaft in the ear so that the temperature within the portion of the ear where the temperature sensor is positioned is in equilibrium, which may be particularly helpful for resistive temperature sensors (such as thermistors) that do not contact the ear. In some variations, the external seating body may be relatively flat and may also seal against the outer ear, providing an additional thermal seal. The external seating body may also be shaped so that it may be comfortably worn, having a low profile (e.g., less than 1 cm thick, less than 0.9 cm thick, less than 0.8 cm thick, less than 0.7 cm thick, less than 0.6 cm thick, less than 0.5 cm thick, etc.). The outer surface of the external seating body may include or may be formed by a soft (low durometer) material, such as silicone (other low durometer materials may include those having a durometer of, e.g., 80-90 Shore A or less). A portion of the external seating body may be covered by a cover or sleeve of soft (low durometer) material, while the majority of the external seating body is a more rigid material (e.g., ABS).

The temperature sensor may be positioned anywhere on the insertion shaft. For example, the temperature sensor may be present at the distal end of the shaft and configured so that it does not contact the ear. Alternatively the sensor may be positioned, e.g., on a side of the insertion shaft, so that it is immediately adjacent to or contacts the ear canal. In general, the earpiece includes a seal around the insertion shaft that is located proximal to the temperature sensor. This seal may plug or otherwise close the ear canal, allowing thermal equilibration of the ear canal where the temperature sensor is positioned. This may allow stable readings from temperature sensor (e.g., thermistor). In addition, the external seating body may also seal the ear canal from the outer ear portion. For example, in variations including a soft, low-durometer cover (e.g., sleeve) the sleeve may be configured to for a seal around the outer ear (e.g., the ear canal) which may also enhance the stable readings.

In general, the earpieces described herein may include a wireless communication circuitry (e.g., Bluetooth); alternatively or additionally the base station may include wireless communication circuitry (e.g., Bluetooth, WiFi, ZigBee, near field communication (NFC), etc.). The presence of wireless communication circuitry in the earpiece may allow the apparatus to operate in real time, e.g., permitting real time monitoring for transmission of data directly to a wearable processor (e.g., Google Glass), a hand-held processor (e.g., smartphone), or remote processor (e.g., laptop, desktop, etc.). In some variations the earpiece does not transmit until after it is stably docked into the base station, as verified by the microcontroller. For example, data (temperature data and/or additional data collected from the earpiece) may be transmitted by the earpiece after docking to the base station to a remote processor (e.g., smartphone).

The earpiece may generally detect when it is docket or undocked from the base station, and may automatically begin recording temperature (without requiring any additional outside control). For example, the apparatus, upon removal from the base station, may automatically detect that it is not connected and may switch from a docked mode (charging, transferring data) into a user (use) mode. In the user mode, the earpiece may run continuously as long as there is sufficient battery charge (e.g., greater than a hibernation battery charge threshold) while undocked, and may switch between a low-power sleep mode and an active, recoding data mode. In the recording data mode, power may be applied to the temperature sensor to record one or more readings (e.g., plurality of readings in quick succession, such as 2-3 samples). If multiple readings are made they may be averaged (while analog if an analog sensor is used) and then converted to a digital signal using the ADC. After storing the digital value in memory, a counter may be incremented (indicating a “wake up” occurred) and the earpiece may then transition back into the low-power sleep mode, where it may remain for the predetermined time interval (e.g., five minutes). Each time the earpiece apparatus “wakes up” it may take and record a measurement as described. Thus, in general, the microprocessor of the earpiece may include a user mode that encompasses a sleep mode during the microprocessor removes power from the majority of the components, including the ADC, temperature sensor, etc. A timer (watchdog timer) may remain running to trigger the automatic wake-up transition to the active mode. When the earpiece wakes up, it applies power to the sensing circuit(s), including the temperature sensor(s), the ADC, etc., and may start measuring a signal from the temperature sensor (e.g., thermistor) and convert the signal into an actual temperature measurement so that the (digital) temperature measurement may be stored in memory. During the active or sleep mode (either or both) the earpiece may detect a low battery signal by comparing the battery charge to a threshold voltage value (hibernation threshold). For example, the hibernation threshold may be 3.2V; when the battery charge falls below 3.2V, the earpiece may shut down, entering into a hibernation mode. After connecting the earpiece to the base station and allowing it to recharge, the earpiece may exit the hibernation mode (into base mode or again into the user mode).

Either or both the earpiece and the base station may include one or more outputs such as LEDs, speakers, etc. For example, the base station may include one or more LEDs configured as indicator lights for indicating the status of an earpiece (e.g., attached to the base station, charging, charged, transmitting data, etc.). The LEDs may be different colors (or may be capable of showing different colors). For example, a blue LED may indicate transmitting data, a yellow/red may indicate battery charge (medium, low, etc.), flashing may indicate activity (e.g., charging, transmitting, etc.).

Charging and transmission of information between the earpiece and the base station may be done through the connectors/contacts as mentioned above. For example, the base station may include multiple connectors (e.g., in some variations, six ‘pins’ such as pogo pins may be used). For example, the pins may correspond to high, ground, reset, triggering and two data pins. Corresponding contacts may be present in the earpiece, and may be configured to be low-profile, such that the pins are recessed or flush with an outer surface of the earpiece. As mentioned, in some variations, the base station does not include a processor, but is controlled by the microprocessor on the earpiece. Thus, the earpiece microprocessor may drive communication, (transmission of data from the stored memory on the earpiece), charging, etc. For example, the base station may include an JO extender that receives commands from the earpiece, which may simplify the system; alternatively both the earpiece and the base station may include a microprocessor.

In general, it may be beneficial to have a stable connection between the earpiece and the base station; without a stable connection, the earpiece may “hang up” and not function properly. In some variations, as mentioned above, the earpiece may confirm that a stable connection has been made with the base station before initiating communication and/or other dock mode activities (e.g., charging, transmission, memory clearing, etc.). For example, the earpiece may determine that a contact has been made by initially sensing a connection between one or more of the contacts on the earpiece and connectors on the base station. Following an initial contact, the microprocessor (or another circuit) in the earpiece may monitor this connection for a predetermined time period before switching the earpiece to dock mode. For example, the earpiece may wait for a confirmation duration (e.g., approximately 0.5 seconds, 1 sec, 2 seconds, 3 seconds, 5 second, 10 seconds, 20 seconds, 30 seconds, 1 minute, 2 minutes, etc.), such as one minute; if, during that confirmation period, the contact between the earpiece and the base station is no interrupted, the earpiece may proceed, having confirmed the quality of the connection. In practice, this delay period (the confirmation period) may allow the user to close the lid, securing the contact by applying force against the earpiece to hold the contact with the base station. After a waiting period (e.g., a minutes worth of cycles) the earpiece may determine that the connection is stable and may then transmit and charge (in the dock mode). The earpiece may continue to monitor the stability of the connection. Although the miniaturization of the contacts and connectors between the earpiece and the base station may make reliable docking more challenging, the use of the locking lid (including the engagement of a portion of the lid with a depression or other portion of the earpiece) may enhance the reliability of the connections.

The data (e.g., temperature data) is generally stored as a digital temperature reading, which may be preferable as it may allow a higher density of data to be stored. In addition or alternately, a custom data structure may be used. For example, the data structure may be compact, so that all of the collected data recorded prior to docking and transmission may be transmitted at the same time.

In addition, any of the apparatuses (e.g., earpiece devices) described herein may also include temperature calibration. Calibration may be done as part of the manufacturing process (factory calibration) and/or by a user. For example, to calibrate a device the sensor portion (or the entire device) may be placed in a temperature controlled liquid (e.g., a water circulation bath kept at known temp). The device may generate an offset based on the value detected and the known value and this offset (+/−) may be added to temperature measurements made by the device. In some variations the apparatus may include a calibration mode, which may be used for factor and/or for self- or user-calibration. In the calibration mode, the user may be instructed to place the apparatus into an ice bath (e.g., a cup or bowl full ice and water). A sealable container may be provided. The apparatus may then detect the temperature, and compare it to the pre-programmed value (temperature) and update the offset value (calibration offset value) that may be added/subtracted to any temperatures detected thereafter. Alternatively or additionally, the base station may include a calibration mode in which the base station applies a known or control temperature (e.g., via a resistive heating element, Peltier-effect device, or the like) when the apparatus is in a calibration mode to determine the calibration offset and thereby calibrate the earpiece.

As mentioned above, any appropriate sensor may be used in addition to the temperature sensor. One or more sensor may also be used to adjust or modify the temperature sensing. For example, the earpiece may include a motion sensor or an inclination sensor that is configured to determine when a user wearing the earpiece is lying on their ear, which may result in a local warming in the ear canal. An angle (inclination) sensor, including an accelerometer may indicate when the earpiece is worn in an ear so that the back (outer) surface of the external seating body is facing ‘down’ relative to gravity. Alternatively or additionally, a pressure or contact sensor may be included on the external seating body which may indicate when the user is lying on the ear in which the earpiece is worn. During operation, the apparatus may record these conditions, which may be correlated with the temperature data and may be used to compensate for temperature readings based on the periods or lengths of time that the user was lying on the earpiece.

In general, described herein are systems for determining basal body temperature, the system comprising: an earpiece having: an insertion shaft extending distally from an external seating body, a temperature sensor within the insertion shaft, a microcontroller, a plurality of electrical contacts electrically connected to the microcontroller, and a seal around the insertion shaft proximal to the temperature sensor so that the temperature sensor is sealed within the ear canal when the earpiece is worn in a user's ear; a base station configured to couple with the earpiece, the base station including a docking cradle having a plurality of connectors configured to connect with the plurality of electrical contacts on the earpiece when the earpiece is seated in the docking cradle; and a non-transitory computer-readable storage medium storing a set of instructions capable of being executed by a processor, that when executed by the processor causes the processor to: receive a plurality of temperature data from the earpiece, determine basal body temperature from the temperature data, and display a representation of the basal body temperature.

A system for determining basal body temperature may include: an earpiece having: an insertion shaft extending distally from an external seating body, a thermistor within the insertion shaft, wherein at least part of the external seating body comprises or is covered in a soft, low-durometer, material, a microcontroller, a battery, a plurality of electrical contacts on the external seating body electrically connected to the microcontroller, and a seal around the insertion shaft proximal to the temperature sensor so that the temperature sensor is sealed within the ear canal when the earpiece is worn in a user's ear; a base station configured to couple with the earpiece, the base station including a docking cradle having a plurality of connectors configured to connect with the plurality of electrical contacts on the earpiece when the earpiece is seated in the docking cradle; and a non-transitory computer-readable storage medium storing a set of instructions capable of being executed by a processor, that when executed by the processor causes the processor to: receive a plurality of temperature data from the earpiece when the earpiece is docked in the docking cradle, calculate time information from the data, determine basal body temperature from the temperature data, and display a graph of the basal body temperature. The external seating body may comprise or be covered in (including as part of a sleeve or cover) a soft, low-durometer, material.

In any of the systems described herein, the earpiece may include an analog-to-digital converter (ADC), and a memory, and the microcontroller is configured to periodically take multiple samples the temperature, average the samples, convert the average from analog to digital and store the average in the memory.

The plurality of electrical contacts may be flush with or recessed into the external seating body. The earpiece may include a depression on an upper surface of the external seating body. The base station may include a lid and latch, wherein the latch is configured to hold the lid closed so that the lid applies pressure against the earpiece when the earpiece is within the docking cradle so that the plurality of connectors maintains electrical contact with the plurality of electrical contacts.

The earpiece may include a thermal insulation between the insertion shaft and the external seating body.

In any of these variations, a user hand-held processor such as a smartphone or table (or wearable electronics) may be used. For example, the non-transitory computer-readable storage medium storing a set of instructions may be configured act on a smartphone processor. The non-transitory computer-readable storage medium storing a set of instructions capable of being executed by a processor may further cause the processor to: correlate a date and/or time with the data. In any of these apparatuses, the non-transitory computer-readable storage medium storing a set of instructions capable of being executed by a processor may further causes the processor to request user information comprising one or more of: menstrual data, sexual activity, ovulation, ovulation test data, vaginal discharge. This information may be used, along with the temperature data (or the temperature data alone may be used) to predict ovulation and/or fertility. For example, the non-transitory computer-readable storage medium storing a set of instructions capable of being executed by a processor may further causes the processor to predict ovulation based on basal body temperature.

Also described herein are systems including an earpiece and a base station that ensures good connection between the two when the earpiece is docked (e.g., before transmitting data and/or charging). Thus, for example, the apparatus may confirm that the device is making a good contact before switching to a docked mode.

For example, a system for determining basal body temperature, the system comprising: an earpiece having: an insertion shaft extending distally from an external seating body, a temperature sensor within the insertion shaft, a microcontroller, and a plurality of electrical contacts on the external seating body electrically connected to the microcontroller, wherein the electrical contacts are flush with or recessed into the external seating body; and a base station, the base station including: a docking cradle having a plurality of connectors configured to connect with the plurality of electrical contacts on the earpiece, a lid, and a latch configured to hold the lid closed so that the lid applies pressure against the earpiece when the earpiece is within the docking cradle so that the plurality of connectors maintain electrical contact with the plurality of electrical contacts, wherein the microcontroller is configured to detect contact between at least one of the electrical contacts and at least one connector for a contact time of greater than 5 seconds before transmitting temperature data from the earpiece.

For example, a systems for determining basal body temperature may include: an earpiece having: an insertion shaft extending distally from an external seating body, a depression on an upper surface of the external seating body, a temperature sensor within the insertion shaft, a microcontroller, a battery, and a plurality of electrical contacts on the external seating body electrically connected to the microcontroller, wherein the electrical contacts are flush with or recessed into the external seating body; and a base station, the base station including: a docking cradle having a plurality of connectors configured to connect with the plurality of electrical contacts on the earpiece, a lid having a protrusion configured to mate with the depression on the external seating body of the earpiece, a latch configured to hold the lid closed so that the lid applies pressure against the earpiece when the earpiece is within the docking cradle so that the plurality of connectors maintain electrical contact with the plurality of electrical contacts, wherein the microcontroller is configured to detect contact between at least one of the electrical contacts and at least one connector for a contact time of greater than 5 seconds before transmitting temperature data from the earpiece.

The lid of the base station may further comprise a protrusion configured to mate with a depression on the external seating body of the earpiece. The plurality of electrical contacts may comprise plates and the plurality of connectors may comprise pogo pin connectors. For example, the plurality of electrical contacts may comprise at least six electrical contacts.

The lid may be hinged on an upper surface of the base station.

Also described herein are earpieces having a low profile that can be comfortable worn over an extended period of time (e.g., all night). These earpieces may be used with any of the apparatuses and methods described herein. For example, described herein are earpiece devices for sensing basal body temperature that is configured to be worn while sleeping, the device comprising: an external seating body, wherein at least part of the external seating body comprises or is covered in a soft, low-durometer, material; an insertion shaft extending distally from the external seating body; a thermistor within the insertion shaft; a seal around the insertion shaft proximal to the thermistor; a microcontroller; and a plurality of electrical contacts on the external seating body electrically connected to the microcontroller, wherein the electrical contacts are flush with or recessed into the external seating body.

An earpiece device for sensing basal body temperature that is configured to be worn while sleeping may include: an external seating body, wherein at least part of the external seating body comprises or is covered in a soft, low-durometer, material; an insertion shaft extending distally from the external seating body; a depression on an upper surface of the external seating body; a thermistor within the insertion shaft; a seal around the insertion shaft proximal to the thermistor so that the thermistor is sealed within the ear canal when the device is worn in a user's ear; a microcontroller; and a plurality of electrical contacts on a lower surface of the external seating body that are electrically connected to the microcontroller, wherein the electrical contacts are flush with or recessed into the external seating body.

The earpiece may include a depression on an upper surface of the external seating body.

In general, the external seating body may have a thickness that is less than 1.5 cm (e.g., less than 1.4 cm, less than 1.3 cm, less than 1.2 cm, less than 1.1 cm, less than 1.0 cm, less than 0.9 cm, less than 0.8 cm, less than 0.7 cm, less than 0.6 cm, less than 0.5 cm, etc.) at its thickest region (excluding the insertion shaft).

As mentioned above, the soft, low-durometer, material may be a soft silicone. The plurality of electrical contacts may comprises plates, and they may be arranged adjacent to each other, on an underside of the external seating body, which faces the user's ear when worn; because they are low-profile (e.g., flat, recessed, etc.) they may be non-irritating. In some variations they may include or may double/act as electrodes (e.g., for detecting electrical signals from the skin). The plurality of electrical contacts may include at least six electrical contacts.

The external seating body may comprise a silicone sleeve or overlay that fits over a portion (e.g., the periphery) of the external seating body.

Also described herein are earpieces that include automatic control of their operation, including multiple modes of operation, such as use/user modes (active, sleep), docked modes (transmitting/charging) and hibernation modes. Thus, the earpiece may be configured in particular to operate robustly while maintaining the battery life for a long period of time without recharging, including greater than 6 hours, 7 hours, 8 hours, 9 hours, 10 hours, 11 hours, 12 hours, 13 hours, 14 hours, 15 hours, 16 hours, etc.

For example, any of the earpieces described herein may be configured to be worn for an extended period of time, the device comprising: an external seating body; an insertion shaft extending distally from the external seating body; a thermistor within the insertion shaft; a seal around the insertion shaft proximal to the thermistor so that the thermistor is sealed within the ear canal when the device is worn in a user's ear; a microcontroller; an analog-to-digital converter (ADC); a memory; a battery connected to the microcontroller; and a plurality of electrical contacts on the external seating body electrically connected to the microcontroller; wherein the microcontroller operates continuously in a user mode by switching at a sampling frequency from a low-power sleep mode, during which no power is applied to the thermistor or the ADC, to an active mode, during which one or more samples is taken from the thermistor, converted to a digital value and the digital value stored in the memory before returning to the low-power sleep mode; further wherein the microcontroller is configured to switch from the user mode into a base mode when the microcontroller detects contact between one or more of the plurality of electrical contacts and a connector of a base station.

An earpiece device for sensing body temperature (including basal body temperature) that is configured to be worn for an extended period of time may include: an external seating body; an insertion shaft extending distally from the external seating body; a thermistor within the insertion shaft; a seal around the insertion shaft proximal to the thermistor so that the thermistor is sealed within the ear canal when the device is worn in a user's ear; a microcontroller; a wireless transmitter; an analog-to-digital converter (ADC); a memory; a battery connected to the microcontroller; and a plurality of electrical contacts on the external seating body electrically connected to the microcontroller; wherein the microcontroller operates continuously in a user mode by switching at a sampling frequency of between once per minute and once per 120 minutes from a low-power sleep mode, during which no power is applied to the thermistor or the ADC, to an active mode, during which a plurality of samples is taken from the thermistor, averaged, converted to a digital value, and the digital value stored in the memory before returning to the low-power sleep mode; further wherein the microcontroller is configured to switch from the user mode into a base mode when the microcontroller detects contact between one or more of the plurality of electrical contacts and a connector of a base station. The microcontroller may be configured to switch the earpiece into a hibernation mode if the battery charge in the earpiece falls below a hibernation threshold (e.g., if the battery charge in the earpiece falls below a hibernation threshold of 3.2 V).

The microcontroller may operate continuously in the user mode by switching at a sampling frequency of every x minutes (where x is 1 min, 2 min, 3 min, 4 min, 5 min, 10 min, 15 min, 20 min, 30 min, 40 min, 45 min, 50 min, 60 min, 90 min, 120 min, etc.) from the low-power sleep mode to the active mode.

The microcontroller, in the active mode, may take a plurality of samples from the thermistor, averages the samples, converts the samples to a digital value and stores the digital value in the memory.

Also described herein are methods of using any of the apparatuses described. For example a method of measuring body temperature (including basal body temperature) over an extended period of time, the method comprising: inserting an insertion shaft of an earpiece into a user's ear so that an external seating body of the earpiece rests within an outer ear region; continuously and periodically switching, at a sampling frequency of between once per minute and once per 120 minutes, from a low-power sleep mode, during which no power is applied to a thermistor or an ADC in the earpiece, to an active mode, during which one or more samples is taken from the thermistor to get a sample value, the sample value is converted to a digital value, and the digital value is stored in a memory in the earpiece before returning to the low-power sleep mode; removing the earpiece from the user's ear; inserting the earpiece into a base station; and switching from the user mode into a base mode when the earpiece detects contact between an electrical contact on the earpiece and a connector on the base station.

A method of measuring body temperature over an extended period of time may include: inserting an insertion shaft of an earpiece into a user's ear so that an external seating body of the earpiece rests within an outer ear region; continuously and periodically switching, at a sampling frequency of between once per minute and once per 120 minutes, from a low-power sleep mode, during which no power is applied to a thermistor or an ADC in the earpiece, to an active mode, during which a plurality of samples is taken from the thermistor, averaged, converted to a digital value, and the digital value stored in a memory in the earpiece before returning to the low-power sleep mode; switching the earpiece into a hibernation mode if the battery charge in the earpiece falls below a hibernation threshold; removing the earpiece from the user's ear; inserting the earpiece into a base station; switching from the user mode into a base mode after the earpiece detects an uninterrupted contact between an electrical contact on the earpiece and a connector on the base station lasting longer than 5 seconds; and transmitting the stored digital values when the earpiece is in the base mode.

The method may include switching the earpiece into a hibernation mode if the battery charge in the earpiece falls below a hibernation threshold. The method may also include transmitting the stored digital values when the earpiece is in the base mode and/or charging the earpiece when the earpiece is in the base mode.

Any of these methods may be used to determine basal body temperature, including using the body temperature data to determine a basal body temperature value over a sleep period or a fixed time period (e.g., spanning multiple hours). For example, any of these methods may include calculating basal body temperature from the stored digital values.

Any of these methods may include continuously and periodically switching between the low-power sleep mode and the active mode at a sampling frequency of every five minutes. The digital values may be stored in the memory without a time and/or date stamp. In some variations the time/date information may be added back to the data by a separate software/hardware/firmware component (e.g., application software).

Any of these methods may include transmitting the stored digital values to a smartphone, wearable electronics, tablet, laptop, etc.

Any of these methods may include determining ovulation based on the stored digital values.

As mentioned above, in any of these methods, the earpiece may confirm that a good connection is made between with the base station. For example, switching from the user mode into the base mode may be done after the earpiece detects an uninterrupted contact between an electrical contact on the earpiece and a connector on the base station lasting longer than a predetermined length of time (e.g., 5 seconds).

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A and 1B show diagrams illustrating how a user may use an ear temperature measuring device within a computer network environment.

FIGS. 2A-2D illustrate cross-sectional diagrams of a user's ear and an ear temperature measuring device when placed within the ear.

FIG. 2E illustrates a cross-sectional diagrams of an ear temperature measuring device when not placed within the ear.

FIG. 3 is a block diagram of an ear temperature measuring device.

FIG. 4 is a flowchart of a method for monitoring a patient's basal body temperature with an ear temperature measuring device.

FIGS. 4A-4C illustrate half cross-sectional diagrams simulating heat sensitivity of an ear temperature measuring device comprising layers of different thermal conductivity and an enclosing layer at given ambient and ear temperatures.

FIGS. 5A-5C illustrate half cross-sectional diagrams simulating heat sensitivity of an ear temperature measuring device comprising none, one or a dual metal sheath at given ambient and ear temperatures.

FIGS. 6A-6F illustrate half cross-sectional diagrams simulating heat sensitivity of an ear temperature measuring device comprising different geometries and layers of different thermal conductivity at given ambient and ear temperatures.

FIGS. 7A-7B illustrate temperature profile measurements using an ear temperature measuring device.

FIG. 8 depicts a typical human sleep process comprising several cycles of NREM-REM stages.

FIGS. 9A and 9B are perspective views showing one variation of an earpiece having a temperature sensor (e.g., thermistor) that may be placed inside an ear.

FIG. 10A is a partially exploded perspective view of a base station and FIG. 10B is a perspective view of a base station for receiving and connecting to an earpiece.

FIGS. 11A and 11B show a flowchart illustrating functional steps that may be carried out by an earpiece, base station and a smartphone.

FIG. 12A is a functional block diagram of an earpiece in a use mode (e.g. active/awake) applied in sensing and measuring the temperature; FIG. 12B is a functional block diagram for transferring data to the base station (in a docked mode).

FIGS. 13A and 13B are functional block diagrams of a base station coupled to an earpiece and communicating with a smartphone for transferring data to the smartphone.

FIG. 14 is a functional block diagram illustrating data exchange between a collector and thermometer implemented in a Bluetooth software operation.

FIG. 15 is an example of an earpiece configured to monitor multiple parameters including body temperature, and one or more of: motion, ambient noise, hart rete, breath rhythm, blood pressure, blood oxygen level. An earpiece may also include a voice notification unit, battery and data processing (microprocessor) and transmitting unit (wireless transmission circuitry).

FIG. 16A-16D illustrate perspective, side, bottom and top views, respectively, or one example of an earpiece as described herein.

FIG. 17 illustrates a cross-section through a midline of the earpiece of FIGS. 16A-16D (as shown by the line 17 in FIG. 16B).

FIG. 18 is an exploded view of the earpiece of FIGS. 16A-16D.

FIG. 19 is a cross-sectional diagram of a user's ear showing the position of an earpiece such as the one show in FIGS. 16A-16D.

FIG. 20 is an illustration of a low-profile earpiece inserted into a user's ear, as shown.

FIGS. 21A-21D show top, perspective, front and back views, respectively of a base station for docking an earpiece.

FIGS. 22A-22D illustrate securely docking an earpiece in a base station as described herein.

FIG. 23 illustrates the inside of a base station such as the one shown in FIGS. 21A-22D, with the upper cover (and lid) removed to show internal detail.

FIG. 24 is a section through the front of the base station of FIGS. 21A-22D.

FIG. 25 schematically illustrates a system including a base station (base unit), earpiece, handheld processor (e.g., smartphone) running application software and a remote server.

FIGS. 26A-26R illustrate user interface (“screens”) of an application software that may be used as part the systems described herein, including exemplary user interfaces. The example shown in FIGS. 26A-26R is configured as basal body temperature monitoring to determine ovulation.

### DETAILED DESCRIPTION

In the following description, numerous specific details are set forth regarding the systems, methods and media of the disclosed subject matter and the environment in which such systems, methods and media may operate, etc., in order to provide a thorough understanding of the disclosed subject matter. It will be apparent to one skilled in the art, however, that the disclosed subject matter may be practiced without such specific details, and that certain features, which are well known in the art, are not described in detail in order to avoid complication of the disclosed subject matter. In addition, it will be understood that the examples provided below are exemplary, and that it is contemplated that there are other apparatuses (e.g., systems and devices), methods and media that are within the scope of the disclosed subject matter.

In general, described herein are earpiece devices configured to detect body temperature and system including such earpieces. These apparatuses may be used to continuously monitor a user's body temperature and other vital signs or environmental conditions for an extended period of time, including during sleep. Thus, in particular, these apparatuses are adapted to provide comfortable devices and systems that may be reliably operated over a long period of time without requiring recharging. These apparatuses may be used to monitor, diagnose and treat a user, including an active (awake) and asleep user. In one non-limiting example, described herein are apparatuses for determining ovulation.

### Ovulation Prediction System

An ovulation prediction system may comprise an ear temperature measuring device (“earpiece”) that is configured to continuously measure a person's body temperature. The Basal Body Temperature (BBT) may be obtained from the measurements on a daily basis (or other periodic basis) by getting the lowest body temperature measured continuously over the night. The measured temperature may be used to record the person's temperature variations (oscillations) during her sleep and use the measured oscillations to predict a woman's ovulation time by comparing it with previously recorded measurements. In particular, the temperature oscillations may be match to a person's sleeping cycle, e.g., marking the beginning and end of the resting time, to increase the likelihood of correctly predicting the ovulation time. By measuring the temperature within the person's ear the measurements are more accurate and less affected by ambient temperature changes, thus more precisely reflecting a person's body core temperature. To facilitate the analysis of the temperature measurement and allow customization of the ovulation prediction, the data can be transmitted to a user computer. The data transmission can be performed via a cable, wirelessly, using Bluetooth, or any other suitable transmission means. The user computer can include any suitable computing device including for example, a desktop computer, a laptop computer, a tablet computer, a smartphone, or a dedicated computing device.

In this embodiment, the user computer executes a software program that receives the measured temperature data and provides an interface to the user for inputting additional parameters for employing an ovulation prediction algorithm. In addition, the software program provides a graphical user interface for displaying the recorded measurements and the predicted ovulation cycle to the user. By integrating the ear temperature measuring device with a user computer, the software may provide the user with real-time updates, allowing for a physician to more readily review the measured data, and for automatic data storage.

In another embodiment, the system comprises additional physiological sensors to measure, e.g., a person's heart rate, pulse, respiration, blood pressure, and oxygenation. In some embodiments, the sensors can include an accelerometer or gyroscope to determine a person's movements. Measuring a person's movements may then be used by the software program to identify the start and end time of the person's sleeping cycle and match the corresponding times with the temperature oscillations during this cycle.

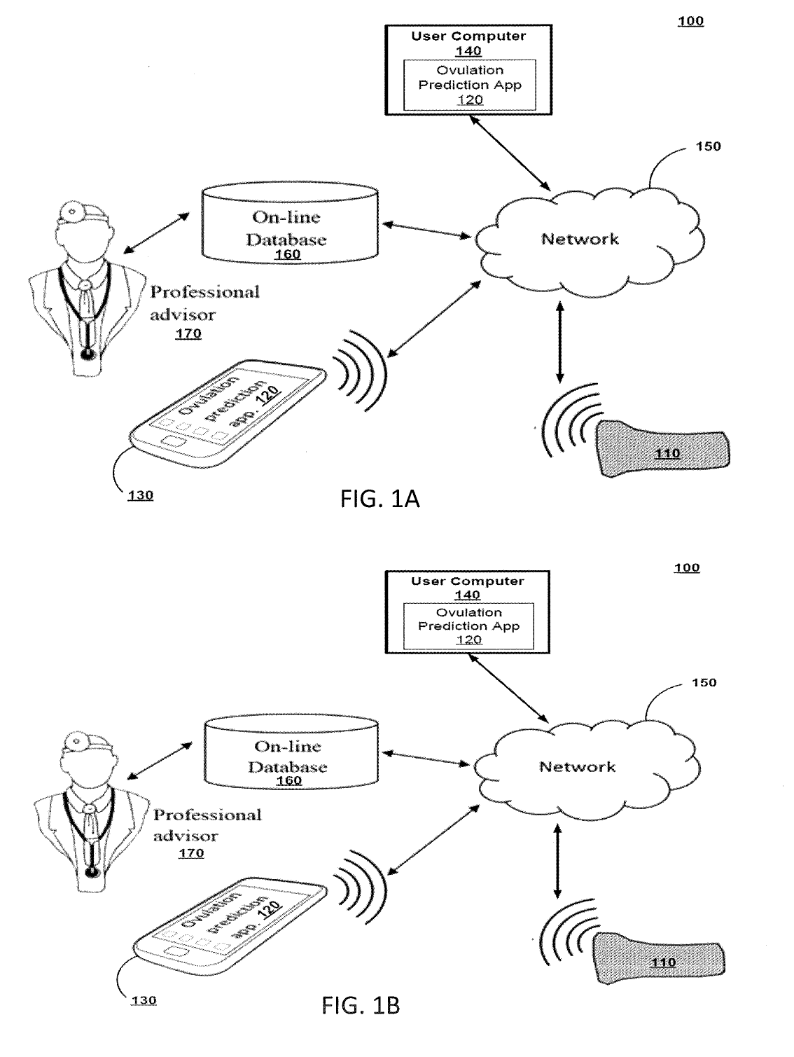

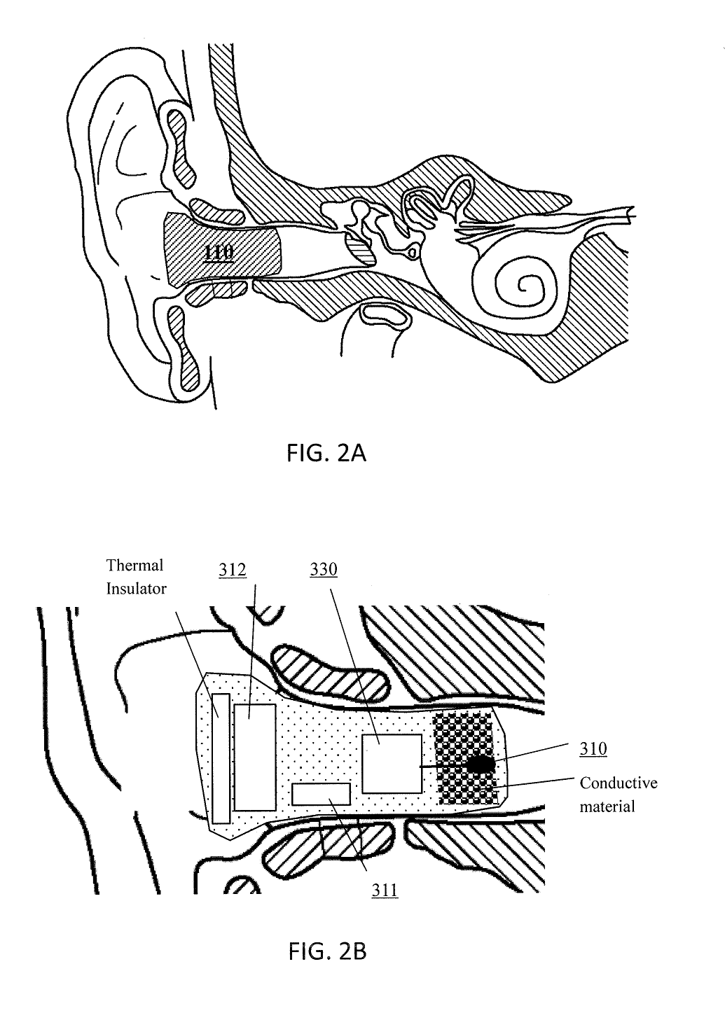

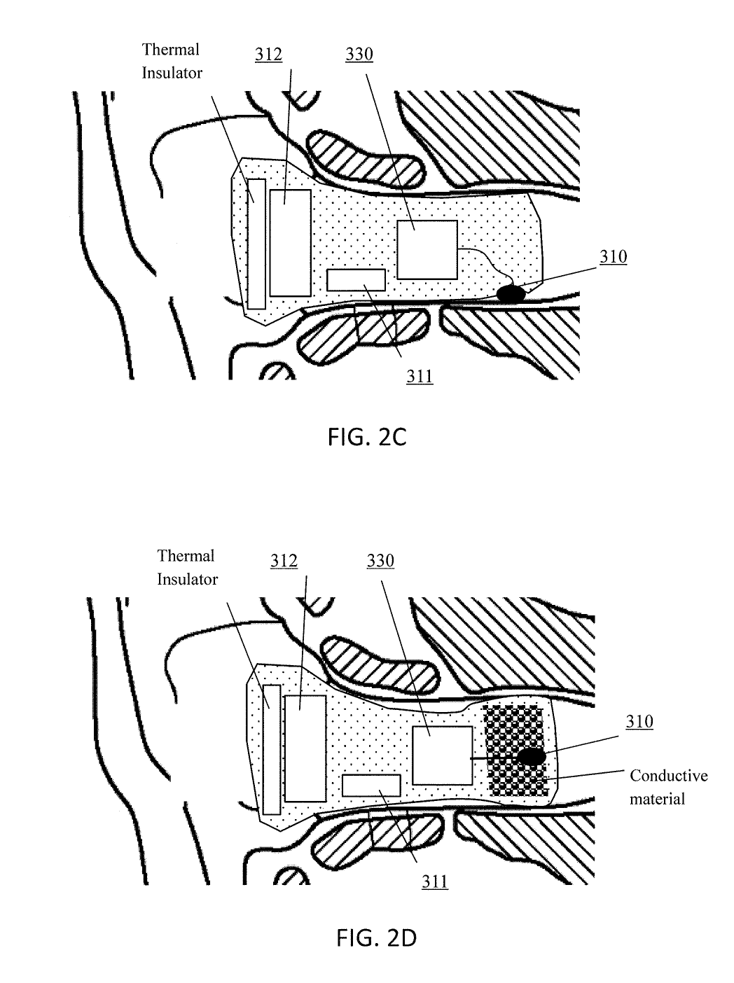

FIG. 1A shows a diagram of an embodiment of an ovulation prediction system 100 that can predict a women's ovulation time by measuring the basal body temperature in the vicinity of the tympanic region of the woman's ear canal. The tympanic region includes the tympanic membrane and the adjacent walls of the ear canal. In one embodiment, the ovulation prediction system 100 comprises an ear temperature measuring device 110, the shape of which closely follows the interior shape of the tympanic region as illustrated in FIGS. 2A-2E. In particular, the ear temperature measuring device 110 can be placed inside the ear to directly contact the surface of the tympanic membrane. FIG. 2C depicts the temperature sensor of the device 110 pressed against the inner skin. In one embodiment, the measuring device 110 comprises a silicon-rubber enclosure to allow for more comfort when wearing the device, thus eliminating the need for any local anesthesia and the risk of damaging the tympanic membrane. In some embodiments, the silicon-rubber enclosure acts as a reliable way of securing the measuring device 110 within the ear canal, preventing its dislodgement when wearing the device over an extended time period or when the patient is sleeping. FIG. 2B shows the structure of an embodiment of the device 110 having extended surface at the tip of the ear plug to assure good pressing against the skin.

Structural embodiments of the ear temperature measuring device 110 include, but are not limited to: (1) the bulk of the device could be made from any type of material (for example, foam/memory foam, silicone, general polymers, thermoplastic, etc.); (2) to reduce heat losses to the surroundings, the device is isolated in the lateral direction (along its axis of insertion into the ear), except at the tip, and is configured to conduct heat from the air cavity in the ear to the temperature sensor; (3) to improve isolation from the ambient, the device might have, near its exterior tip, a region with conductivity below a defined threshold, e.g., existing thermal insulators or a void filled with air with dimensions and structure that precludes internal convection; (4) to assure good contact with the skin, the device 110 is configured to directly pressed against the skin, and/or its structure has a larger diameter at its tip to assure good contact at that region (in this embodiment, the additional structure is made from soft material to assure comfort to the user); (5) the device 110 has a generic fit with different sizes, or custom molded (laboratory made or formed in place) to fit a user's ear and ear canal, and the device 110 being flanged or not flanged, while using different ways of fixture, for example a hook-like structure behind the ear; and (6) device is configured to entirely and/or partially reduce noise and/or external sounds.

In some embodiments, the measuring device 110 is enclosed by biocompatible material, including biocompatible polymers or any other suitable material, to minimize any potential allergic reaction and allow the patient to wear the device for extended time periods. A biocompatible material is either a synthetic or natural material that is not recognized by a body's immune system as a foreign object, thereby evading the immune system's detection and acting as a stealth layer for the measuring device 110. In other embodiments, the outer layer of the ear temperature measuring device is hermetically sealed to prevent water from entering the interior of the device, allowing the device 110 to be washable. Other materials besides silicone rubbers can be used, including, but are not limited to thermoplastics.

Another embodiment of the ear temperature measuring device 110 includes one, two, or more temperature sensing elements or other physiological sensors. A temperature sensor element of 110 includes, but is not limited to: thermistor, thermocouple, thermopile, NTC/PTC, or any other resistance temperature detector (RTD). Alternatively, the temperature sensor element is an infra-red (IR) sensor. To improve accuracy of measurement, an embodiment of the device 110 includes a heating element. The benefit of using a thermistor, instead of an IR sensor, includes that its temperature measurements are not affected by wax buildup inside the user's ear as compared to IR sensors. Embodiments of the ear temperature measuring device 110 can be configured to measure: (1) the body temperature, including BBT, could be made by measuring the temperature of the air inside the ear canal; (2) the temperature of the skin surface in contact with; (3) and/or the temperature of the eardrum.

Other physiological sensors embedded in the ear temperature measuring device 110 include, but are not limited to: (1) an accelerometer and/or gyroscope that senses user movements during the night; (2) pulse oximetry sensor; (3) a brain wave activity sensor (for example, EEG) to measure brain wave activity that directly correlates with sleeping quality; (4) other measuring devices that surrogate signals related to sleeping quality and brain activity, such as sensors for blood pressure, respiration, and oxygenation.

The ear temperature measuring device 110 is also made of conductive material that is used to improve the thermal conductivity between the inner ear skin and the temperature sensor. Conductive material used for the device 110 may include an anisotropic material with radial high conductivity and lateral low conductivity. Furthermore, the material may have special structural characteristics such as windings of small in diameter conductor. In case this material is rigid, e.g., the material being copper, the windings could provide mechanical flexibility while maintaining high conductivity in that region.

The ovulation prediction system 100 further comprises an ovulation prediction software 120 that may run on a smartphone 130 or a user computer 140, all communicatively coupled through a communications network 150 (e.g., the Internet or a wireless network) with the ear temperature measuring device 110. For example, the ear temperature measuring device 110 may be programmed to communicate with the smartphone 130 using a networking protocol such as transmission control protocol/internet protocol (TCP/IP) or any other suitable protocol. Although only one of each type of computing system is shown, in practice many of each type of computing system exist on the Internet, and the various instances of each type of computing systems interact with each other on a frequent basis.

In one embodiment, a user uses a computing device configured to run an ovulation prediction software application 120 to receive the temperature data measured by the ear temperature measuring device 110. For sake of clarity, reference to a user is a reference to the user's computing device, as a mechanism of abstracting away from the actual human actor controlling the computer. Each computing device may include conventional components of a computing device, e.g., a processor, system memory, a hard disk of solid state drive, input devices such as a mouse, a keyboard or touch screen, and/or output devices, such as a monitor or display.

The computing device comprises one or more client devices that can receive user input and can transmit and receive data via the network 150. For example, the client devices may be desktop computers 140, laptop or tablet computers (not shown), smartphones 130, personal digital assistants (PDAs, not shown), or any other device including computing functionality and data communication capabilities. The client devices are configured to communicate via network 150 with the ear temperature measuring device 110, which may comprise any combination of local area and/or wide area networks, using both wired and wireless communication systems. In other embodiments, the ovulation prediction system 100 may include additional, fewer, or different components for various applications. Conventional components such as network interfaces, security mechanisms, load balancers, failover servers, management and network operations consoles, and the like are not shown so as to not obscure the details of the system.

The embodiment in FIG. 1A also includes an online database 160 that stores the data measured by the ear temperature measuring device 110 and the analysis data computed by the client devices 130, 140. In this embodiment, the ovulation prediction systems 120 stores data generated by the ear temperature measuring device 110 in an online database 160 and may communicate data to physician or other medical staff who then may assist the system in correctly predicting the time of ovulation.

FIG. 1B shows a similar diagram with similar operation and functionality as FIG. 1A, but is directed to an embodiment of a sleep quality system 101.

### Configuration of the Ear Temperature Measuring Device

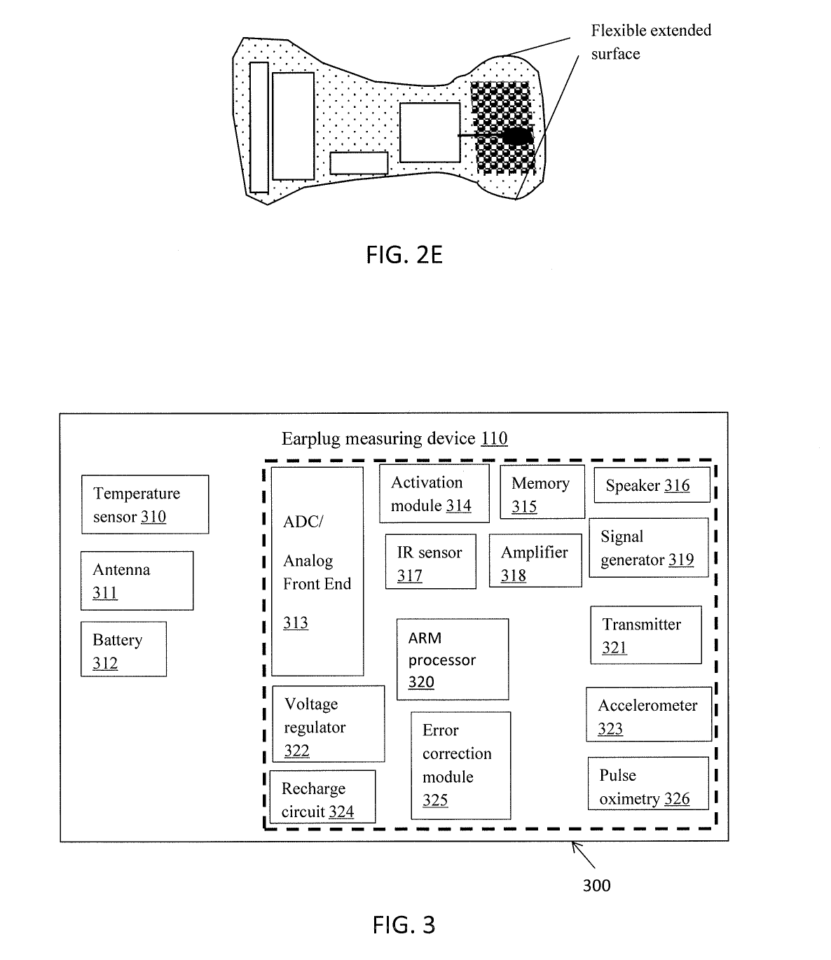

The diagram in FIG. 3 shows components of the ear temperature measuring device 110 according to one embodiment. In this embodiment, the device 110 includes an activation module 310, a sensor 320, and an error correction module 330. The device 110 may also include a processor, a system memory and a power supply, and may be configured to generate a data signal by the signal generator 370 to be transmitted via the communication network 150 to the user's smartphone or computer. In alternative configurations, different and/or additional modules can be included in the device 110. Other electronic components may include: an ARM CPU; an analog to digital converter (ADC); an analog front end chip (AFE); an voltage divider; non-volatile memory to store measurements and to allow asynchronous reads via RF data transmission; a rechargeable or non-rechargeable battery; a voltage regulator circuit; a RF transmitter, receiver and antenna, e.g., Bluetooth, Wi-Fi and NFC; a battery recharging circuit configured for wired or wireless recharging; a miniature speaker for providing feedback to the user potentially used in combination with a Smart Alarm feature; and/or any other suitable component or combination of components. A Smart Alarm feature may be based on the monitored body temperature profile, and could be activated to awake the user at an optimal times (for example, possibly at the high peaks of the temperature which could be related to more conscious stages of the user). The alarm could be active through a miniature speaker inside the device, or it can trigger an external alarm such as, for example, a smart phone.

### Activation of Ear Temperature Measuring Device

In one embodiment, the activation module 310 of the ear temperature measuring device 110 continuously monitors the temperature. Once the device 110 inserted into an ear, the activation module 310 senses an increase in the measured temperature that exceeds a user-specified threshold level with the measured value approximating the body temperature. This threshold level can be set to a value significantly higher than the ambient temperature of storage of the device, which is usually below 35° Celsius. The threshold level could be set at 35.5-36.0 Celsius, or any other suitable Celsius or Celsius range, which is within the spectrum of the normal human body temperature. Once module 310 senses the threshold level, the device starts by first transmitting the stored measurements to the receiver such as a computer or smartphone, or directly go into taking measurements mode. Alternatively, once the device senses a decrease from the level of measured basal body temperature, a decrease which might be as low as ˜1° Celsius or any other suitable Celsius, and thus happen relatively fast after removing the device from the ear, the device is triggered to transmit the collected data to the said receiver, and is triggered back to stand-by mode. If the data was transferred in prior stage, the device goes to stand-by mode directly.

In another embodiment, the device triggering is made by sensing the capacitance and/or resistance of its surroundings. In case resistance is measured, the device includes a couple of electrical contacts to close an electric connection loop through the skin.

In yet another embodiment, if the device is coupled with a sensor for monitoring EEG signals, the device could be operated in the some mode as described in A, only here the device triggering is made by sensing the EEG signal.

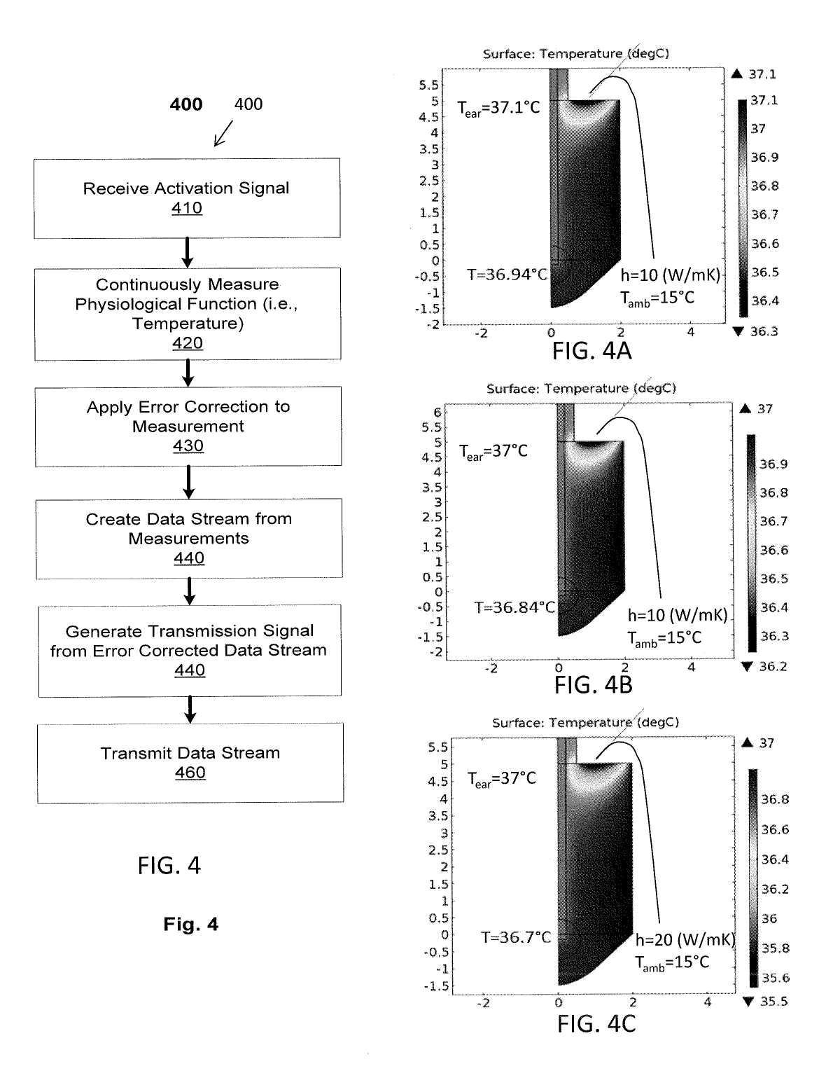

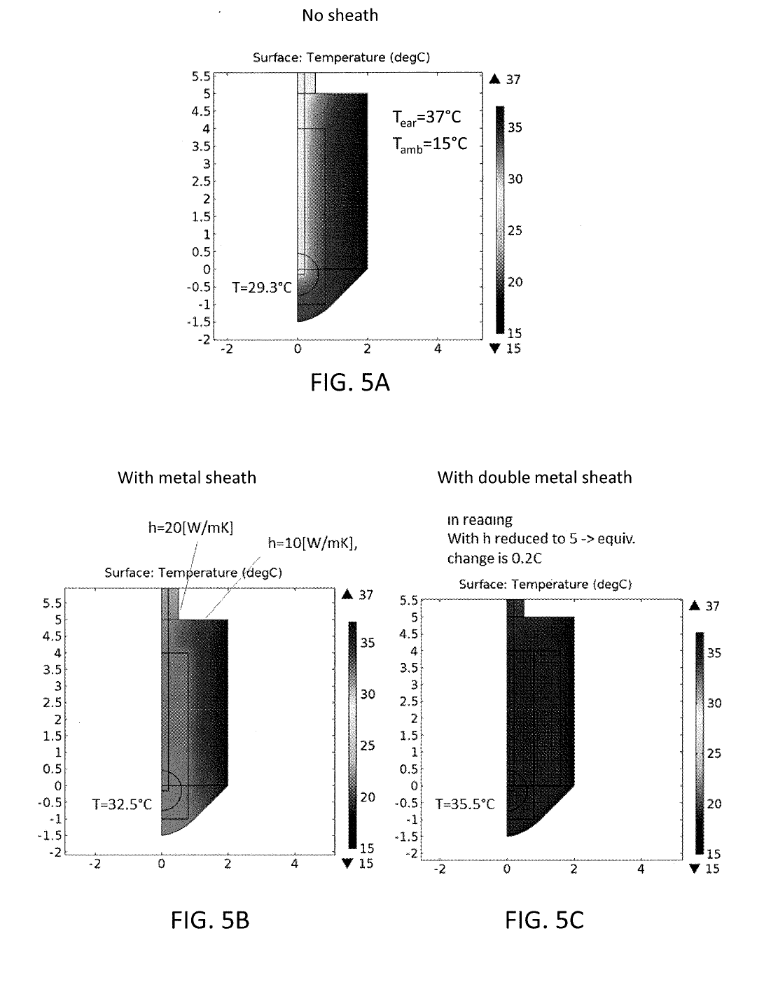

The flowchart in FIG. 4 illustrates a method for monitoring a patient's basal body temperature with an ear temperature measuring device 110, in accordance with one embodiment. The first step in monitoring the basal body temperature comprises activating of the device 110 by the activation module 310.

### Temperature Sensor

The sensor 320 of the ear measuring device 110 comprises a thermistor or temperature transducer. In one embodiment, the sensor 320 comprises one or more thermistors.

### Physiological Sensors

In one embodiment of FIG. 3 the ear temperature measuring device 110 comprises multiple sensors that can measure a person's physiological functions. These sensors include but are not limited to physiological sensors to measure, e.g., a person's heart rate, pulse, respiration, blood pressure, and oxygenation. In some embodiments, the sensors comprise an accelerometer or gyroscope to determine a person's movements.

FIGS. 4A-4C illustrate half cross-sectional diagrams when simulating heat sensitivity of an ear temperature measuring device that comprised layers of different thermal conductivity and an enclosing layer of silicon rubber at a given ambient and ear temperature.

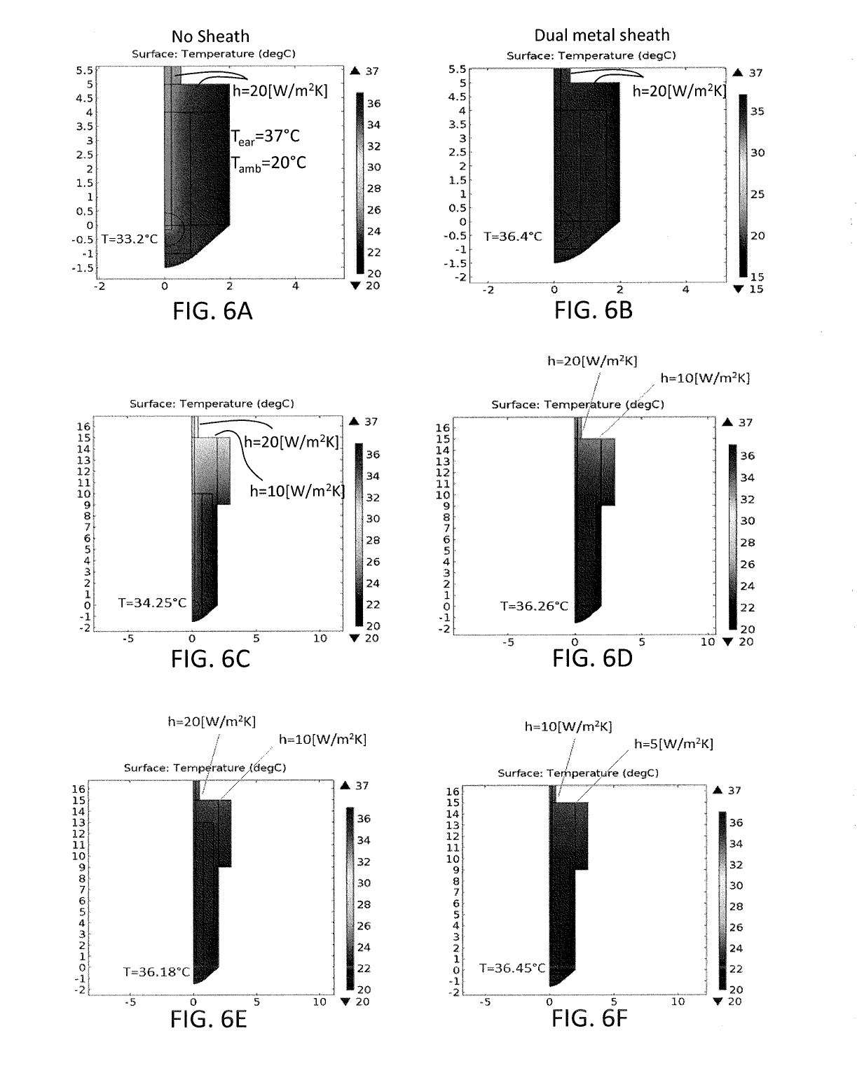

FIGS. 5A-5C illustrate half cross-sectional diagrams simulating heat sensitivity of an ear temperature measuring device that comprised no, one or a dual metal sheath at a given ambient and ear temperature.

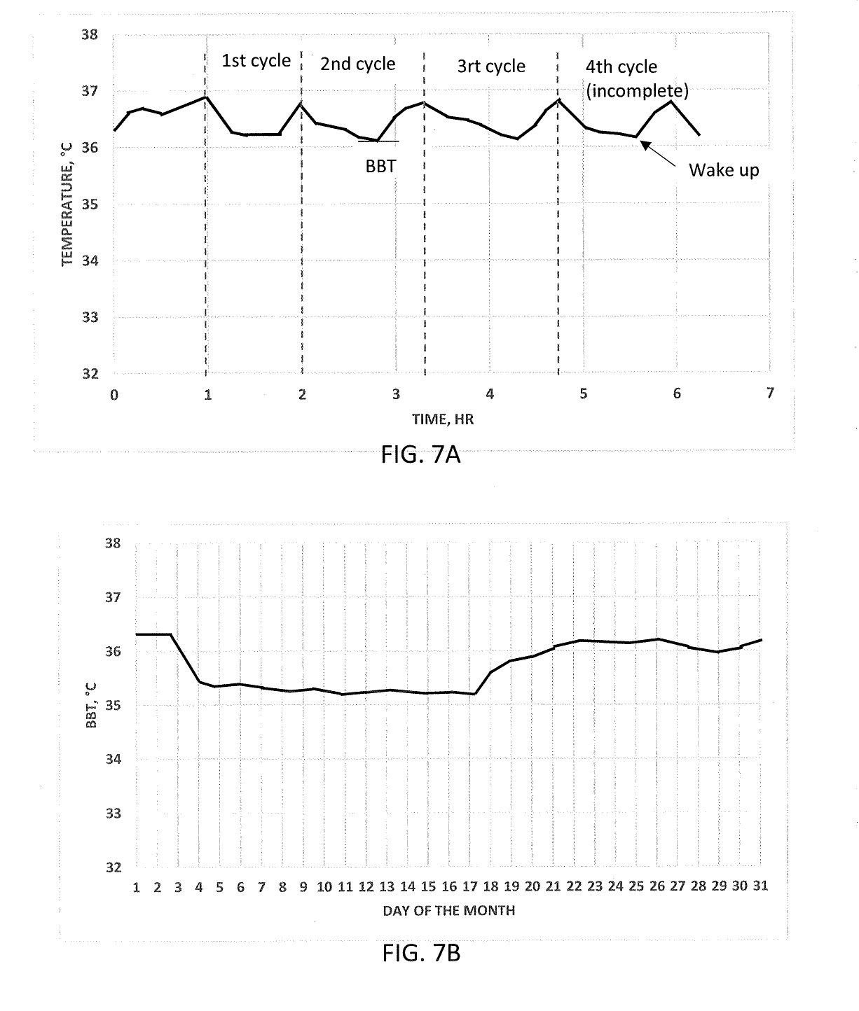

FIG. 6A-6F illustrate half cross-sectional diagrams simulating heat sensitivity of an ear temperature measuring device that comprised different geometries and layers of thermal conductivity at a given ambient and ear temperature.

Influence of ambient conditions: FIGS. 4A-C demonstrate the sensitivity of the measurement on the BBT temperature change. A rise of 0.1° Celsius in the BBT results in the same rise of the measured temperature. FIGS. 4B-C demonstrate the sensitivity of the measurement on increased air flow over the ear of the user. A rise in heat convection coefficient of 10 W/mK resulted in 0.14° Celsius shift in the measured temperature.

Influence of the addition of a highly conductive material/thermal mass (e.g., metal sheath): FIGS. 5A-C demonstrate that, for the case where a thin wire extends away from the device into the ambient, the addition of a highly conductive material greatly improves the accuracy of the measurement and reduces the influence of the ambient conditions on the measurement.

Influence of the addition of a highly conductive material/thermal mass (e.g., metal sheath): FIGS. 5A-B demonstrate that, for the case where a thin wire extends away from the device into the ambient, the addition of a highly conductive material greatly improves the accuracy of the measurement and reduces the influence of the ambient conditions on the measurement. The presented case is for the case where the heat convection coefficients form the prototype's wire and exposed end is 20 W/mK and 10 W/mK, accordingly.

Influence of the addition of a highly conductive material/thermal mass (e.g., metal sheath)—For a different form factor (actual wired prototype geometry): FIGS. 6A-F demonstrate that, for the case where a thin wire extends away from the device into the ambient, the addition of a highly conductive material greatly improves the accuracy of the measurement and reduces the influence of the ambient conditions on the measurement. The presented case is for several possible ambient conditions, defined by the various heat convection coefficients, and a constant ambient temperature of 20 degrees Celsius.

As concluded from the case which is depicted in FIG. 6E, an optimal gap should be kept between the conductive material/thermal mass and the exposed tip of the plug, bellow which excessive heat loss to the ambient occurs and the measured temperature becomes less accurate.

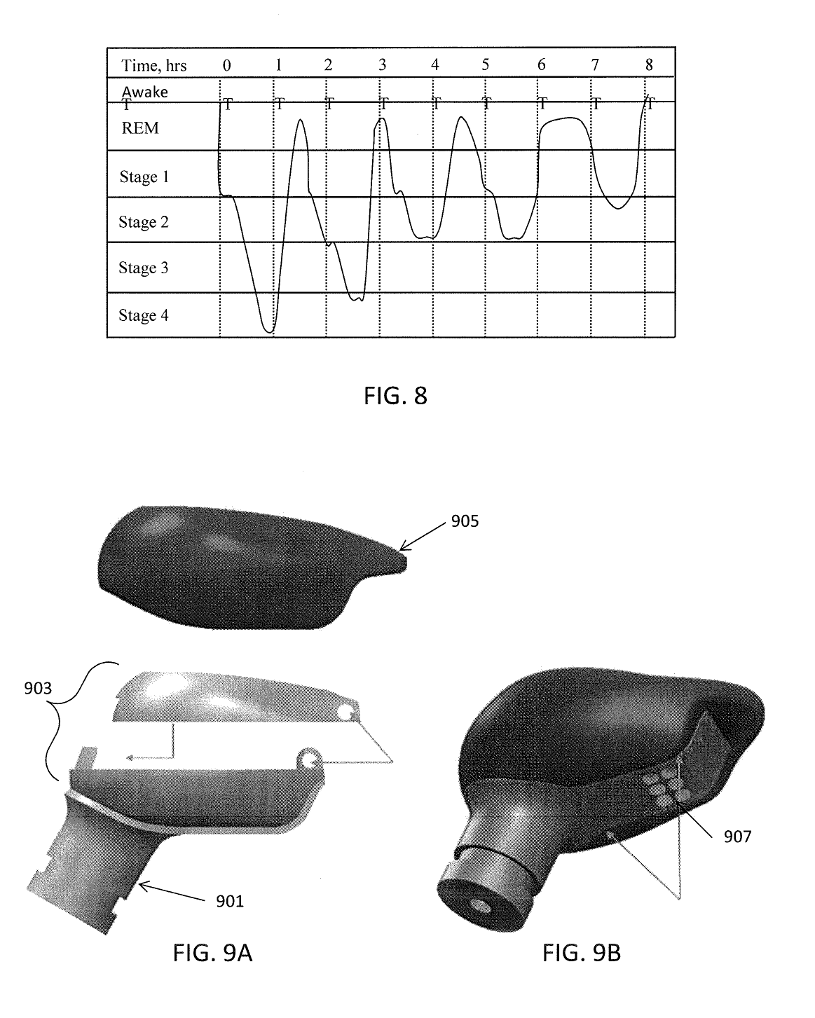

FIGS. 7A-B illustrate examples of temperature measurements using an embodiment of the ear temperature measuring device 110.

In some embodiments, the ear temperature measuring device 110 is configured to measure the following:

### Sample Data

It may be noticed that the temperature fluctuates during sleep, consisting of several cycles. The lowest reachable temperature is considered as the basal body temperature. The duration of the different cycles varies, and so is their amplitude.

Measuring Basal Body Temperature (BBT) based on the measured temperature profile, the BBT is obtained on a daily basis:

The form of the BBT profile and its values carry information that relates to ovulation, sleep quality, cancer and thyroid disease.

For ovulation prediction: BBT rises after ovulation by approximately 0.3 to 0.6° Celsius (or even 1.5° Celsius). A woman is assumed to have ovulated after observing 3 consecutive days of temperature elevation. BBT predicts the peak of fertility, helping a couple to plan the optimal time for coitus. The fertile interval ends on the fourth morning after peak day.

The BBT method could be combined with other user-inputs, such as calendar calculations, period, mucus changes, etc.

For contraception: A couple should refrain from coitus from the first day of a menstrual period until the third consecutive day of temperature elevation above BBT baseline.

As the accuracy of the BBT monitoring method strongly depends on the quality of sleep, and requires at least 4-6 hours of uninterrupted sleep the preceding night, the quality of sleep itself should also be measured to conclude whether the measurement is reliable.

### Measuring Sleep Quality

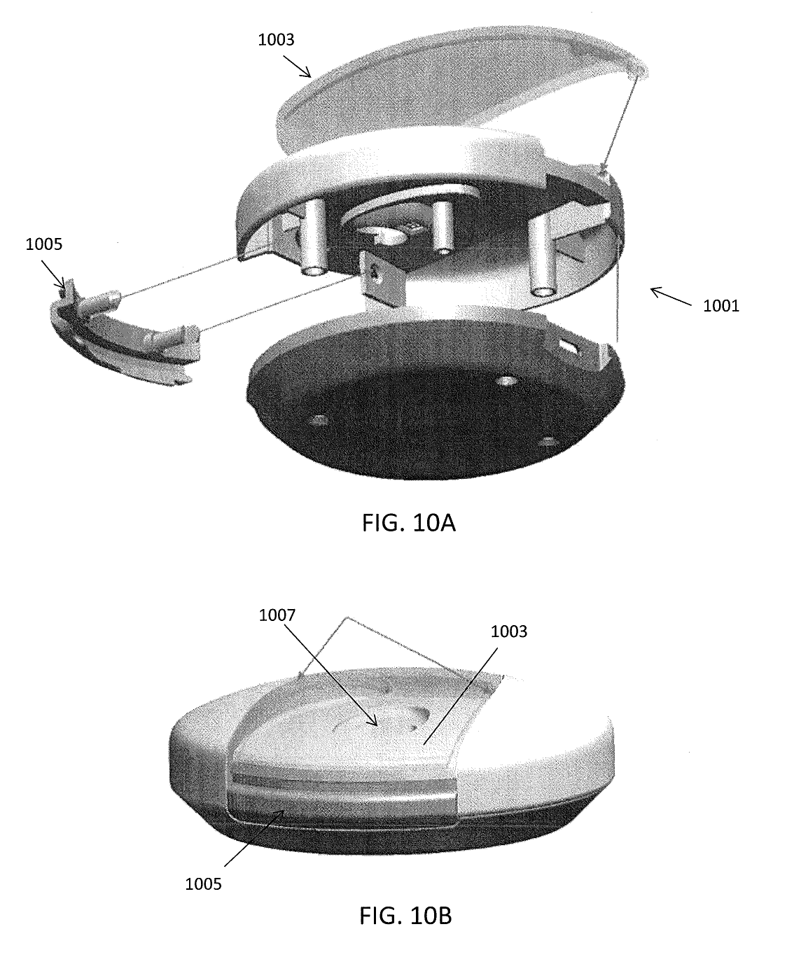

FIG. 8 depicts a typical, 8 hours long, sleep cycle. Looking at the figure, the sleeping process consists of several cycles, where in each cycle the user goes through different consciousness levels. As these cycles relate to the body temperature fluctuations, the number of temperature fluctuation cycles, their duration, and their amplitude, could provide valuable information about the sleep cycle.

The captured temperature profile should by itself provide valuable data about the quality of sleep, but it also may be combined with other sensors such as accelerometer, gyroscope, oximetry, pulse, or any other biometric sensor as described in the product description.

### Smart Alarm Feature

Based on the monitored body temperature profile, a smart alarm could be activated to awake the user at an optimal time (For example, possibly at the high peaks of the temperature which could be related to more conscious stages of the user).

The alarm could be active through a miniature speaker inside the device, or it can trigger an external alarm such as, for example, a smart phone.

### Other Disease Diagnosis

The device can also be used by patients with other diseases that could be correlated to BBT and provide diagnosis features. The device and/or method aim at measuring the core body temperature which could be used in the following applications: ovulation identification & prediction/contraception based on the basal body temperature; sleep quality monitoring based on the temperature fluctuations while sleeping (the later could be also combined with a Smart Alarm feature which is set to waking the user at the optimal timing in the sleep cycle); provide valuable data for Cancer, Hypothyroidism, or Hyperthyroidism patients by comparing their Basal Body Temperature with that of healthy people.

The device operates by any combination of the following functionalities: sensing the temperature in the ear canal (in particular, observing the temperature fluctuations); sensing the heart beat rate/pulse; sensing blood pressure; sensing other signals.

For monitoring sleep quality or other: the temperature fluctuations in the continuously measured temperature profile, possibly including the number of cycles, their duration and amplitude, etc. In case of a value of one or more of the measurements exceeds a set threshold, the system alerts the physician/patient.

As a baby temperature monitor: many parents are having trouble continuously measuring their baby's body temperature and it may be dangerous for an infant to have a fever, particularly for a protracted time period. The apparatuses descried herein may be adapted for monitoring an infant's temperature, and may thus be configured as a baby temperature monitor, which may continuously send the signals, via cable, Wi-Fi, Bluetooth, or other medium, to the parents' computing devices. This will save the parents' efforts of periodically and continually measuring the baby's body temperature (e.g., every 30 minutes, 1 hour, or any other suitable frequency), especially at night. Is the baby removes the apparatus, it may send a warning to the parents' computing devices, and the parents could go to the baby immediately to handle the issue.

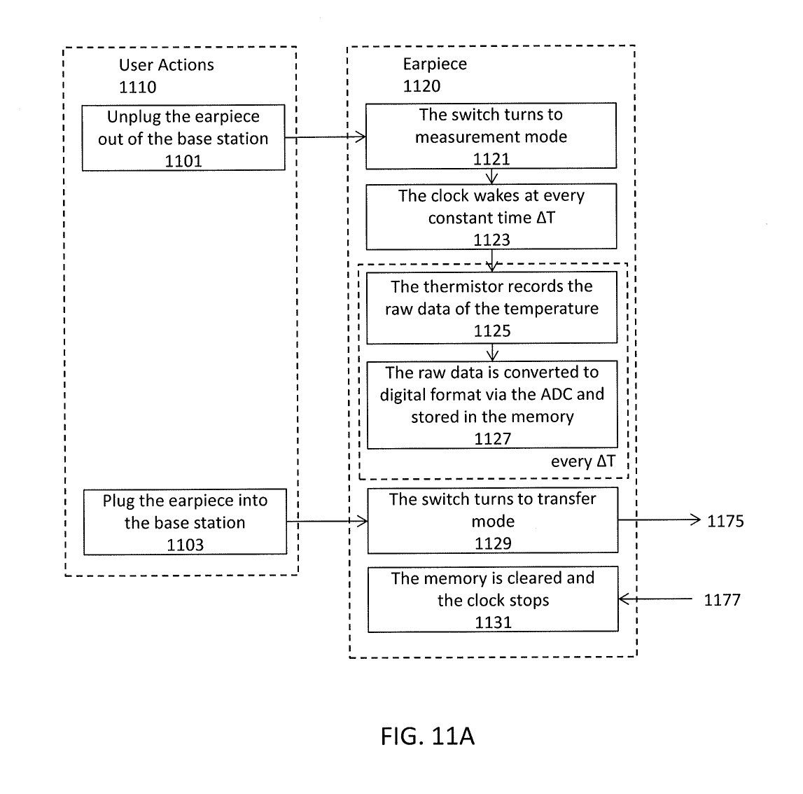

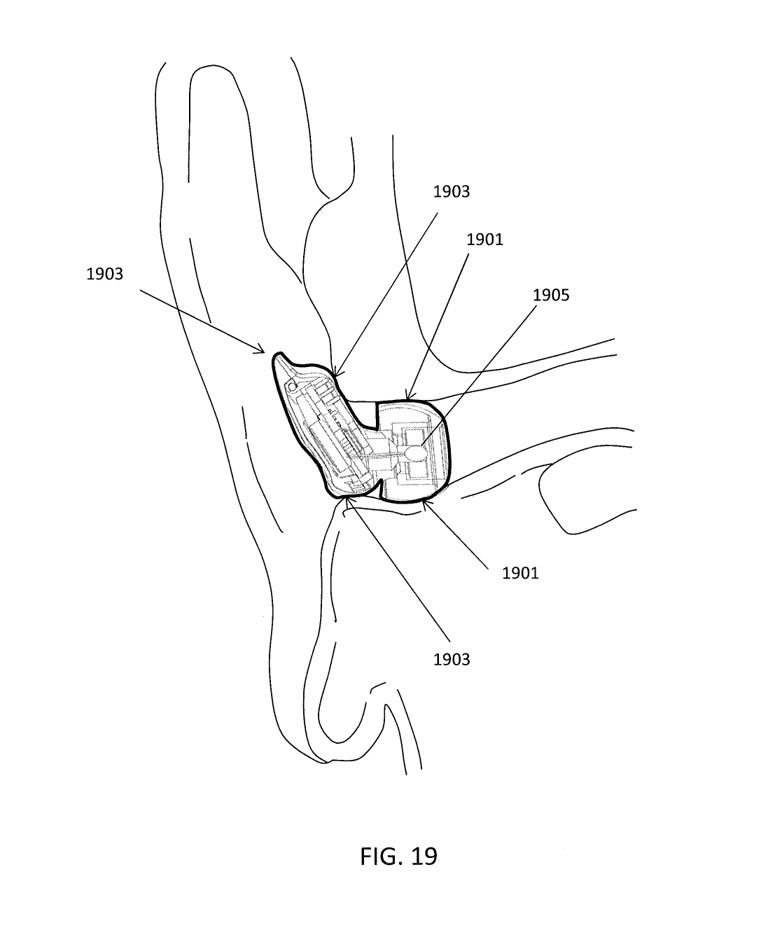

FIGS. 9A and 9B show perspective views to illustrate the shape and structure of one example of an earpiece that may be configured as a temperature sensing device to be placed inside an ear. In FIGS. 9A and 9B, the earpiece includes an insertion shaft portion 901 extending distally from an external seating body 903; a soft outer sleeve 905 may cover or form part of the external seating body. In FIG. 9A, the exploded view shows that the external seating body may be formed in parts including an upper surface region; the control circuitry (e.g., microcontroller, battery, ADC, timer, and additional sensor) may be housed within the external seating body and/or the insertion shaft 901. FIG. 9B shows the assembled earpiece including the exposed thermistor 911 and low-profile contacts 907 on an underside of the external seating body (e.g., a plurality of electrical contacts that are electrically connected to the microcontroller). Other examples of earpieces are shown in FIGS. 16A-16D, 17 and 18.

For example, in FIG. 16A the earpiece is shown in a perspective view and includes an insertion shaft 601 extending distally from an external seating body 605. The external seating body includes a depression 609 in the upper surface. A seal 603 is attached to the insertion shaft 601. A thermistor 612 (not visible in FIG. 16A or 16B, but visible in FIG. 16C) is centered at the distal end of the insertion shaft. FIG. 16B shows a side view of the external seating body 605, insertion shaft 601 and seal 603. The seal 603 is positioned around the insertion shaft 601 proximal to the temperature sensor (thermistor 612). The external seating body shown in FIG. 16B also includes an outer cover comprising a soft (e.g., low durometer) material such as silicone. The lower face (bottom, ear-facing side) of the external seating body includes an indented region 613 at one end, which may provide purchase on the earpiece when removing it.

As shown in FIG. 16C, the underside (bottom) of the external seating body may also include a plurality of electrical contacts 615 on the external seating body that are typically electrically connected to the microcontroller. The electrical contacts 615 may be flat, smooth contacts that are flush or recessed (as shown in FIG. 16C) with the outer surface of the external seating body. FIG. 16D shows a top view, illustrating the indentation 609 or depression (concave region, sized to fit a finger) on the upper side of the external seating body. The external seating body is named as it is because it is the portion of the earpiece that typically sits on the external portion of the ear while the insertion shaft is inserted into the ear canal.

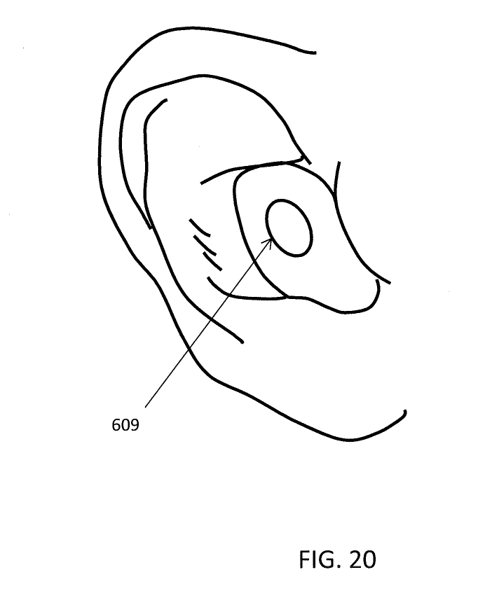

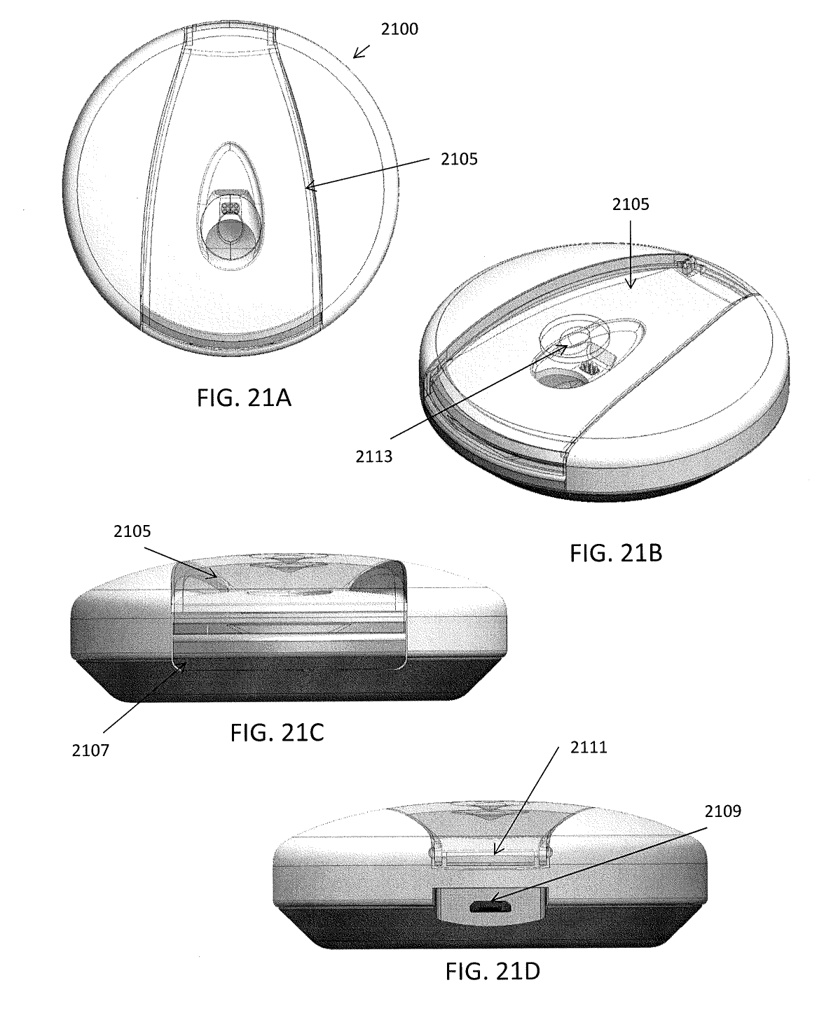

FIG. 17 shows a section through the midline of the earpiece, showing an exemplary arrangement of internal structures within the earpiece, including a temperature sensor (shown as a thermistor 709). The temperature sensor projects from the distal end of the insertion shaft 601, and is sealed within an enclosed portion of the ear canal by a seal 603 formed of a thermally insulating material. In FIG. 17, the thermistor is connected to the internal circuitry, including printed circuit board 721 holding the microcontroller, ADC, memory and also supporting the battery 719. FIG. 18 shows an exploded view illustrating the assembly of an earpiece such as the one shown in FIGS. 16A-17. For example, as shown in FIG. 18, the body of the earpiece is formed of two rigid shells 805, 801 which form the inner shaft and external seating body. The thermistor 709 and control circuitry 721 and battery 719 are all housed within the shell, a soft sleeve 815 attaches over the external seating body, and a seal 603 attaches around the insertion shaft.

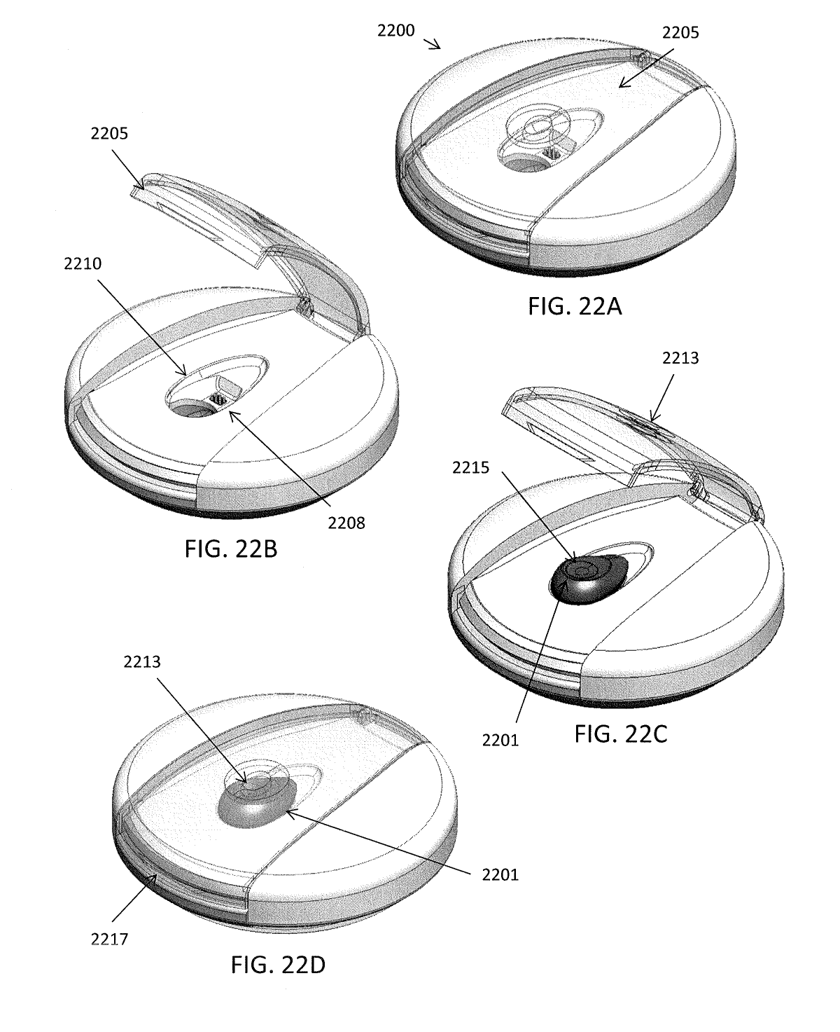

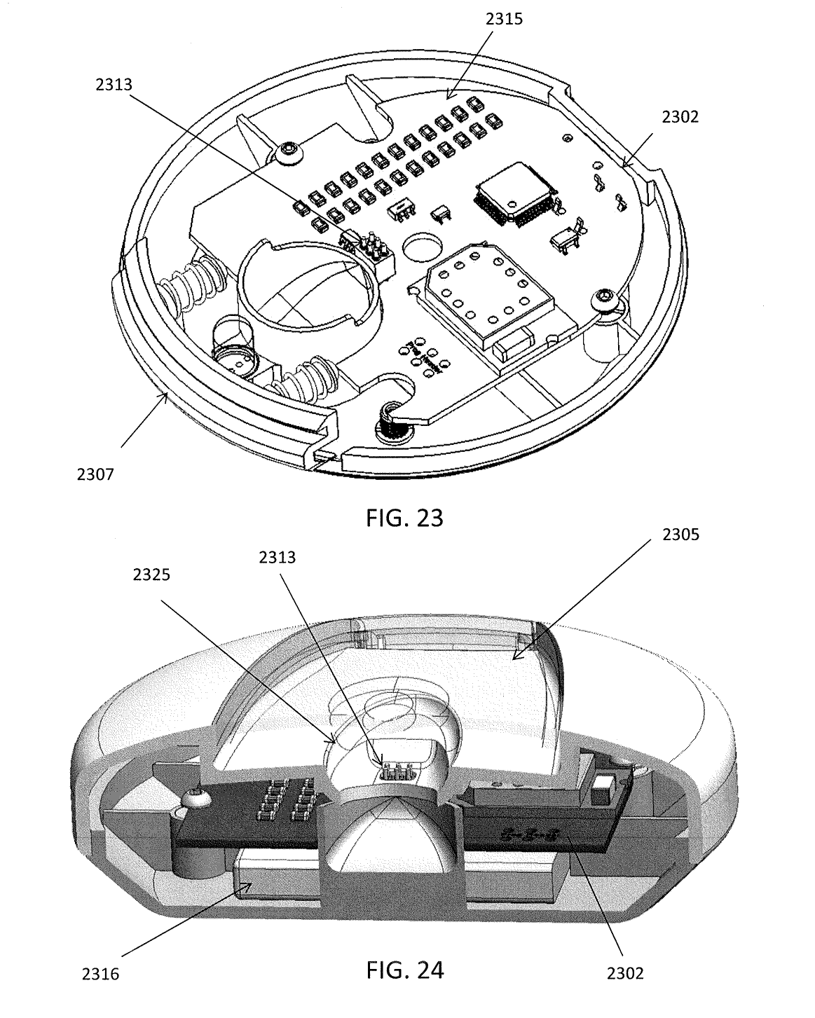

FIGS. 19 and 20 illustrate an exemplary earpiece inserted into a user's ear. In FIG. 19 the earpiece is shown with the distal end of the insertion shaft within the ear canal so that the seal is positioned around the insertion shaft proximal to the temperature sensor 1905; the temperature sensor is thereby sealed 1901 within the ear canal around the wall of the ear canal in an internal region that may readily equilibrate to the temperature of the body. In this example, the external portion of the earpiece also forms a seal on an external portion 1903 of the ear. In general, the earpiece is relatively flat within the outer ear, to prevent being dislodged or becoming uncomfortable during sleep. In the variation shown in FIG. 19, the end of the external seating body having an indented region 1913 that may be used to pull the earpiece out of the ear.

FIG. 20 shows an external perspective view, showing the earpiece within the ear, including the depression/indentation 609 on the upper surface. The external seating region is shown sitting in the concha region of the outer ear.

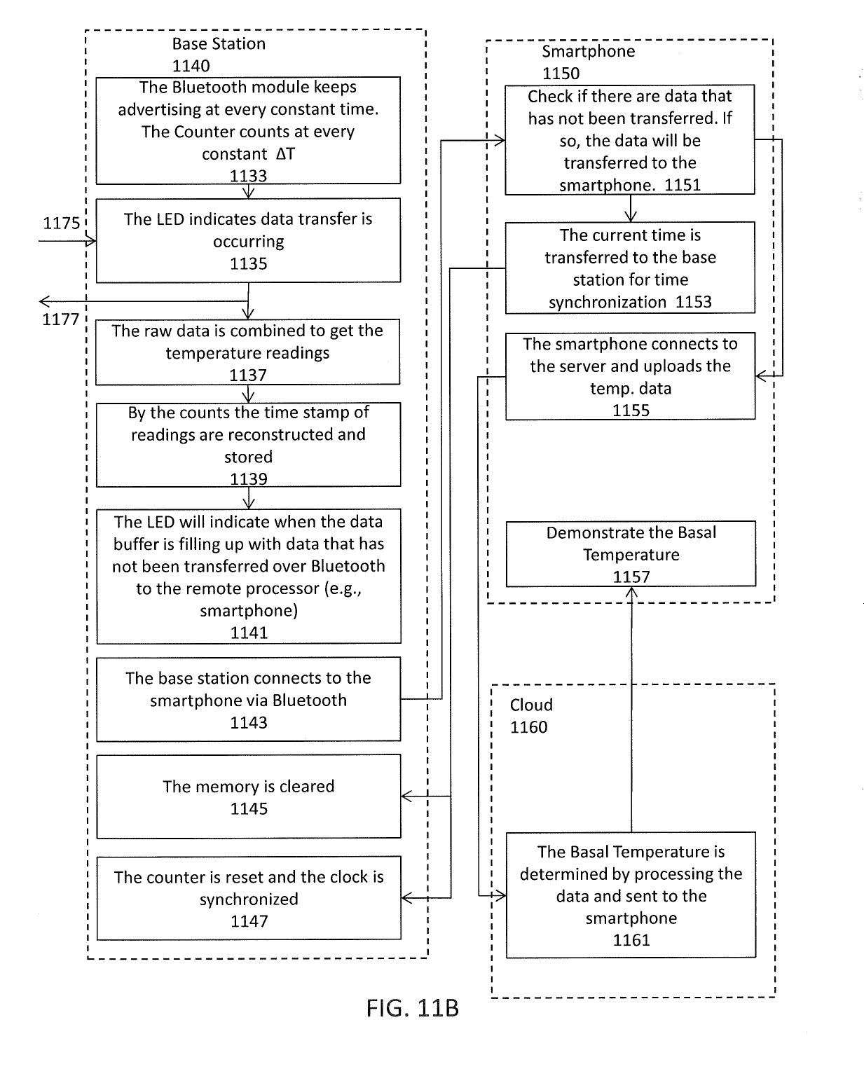

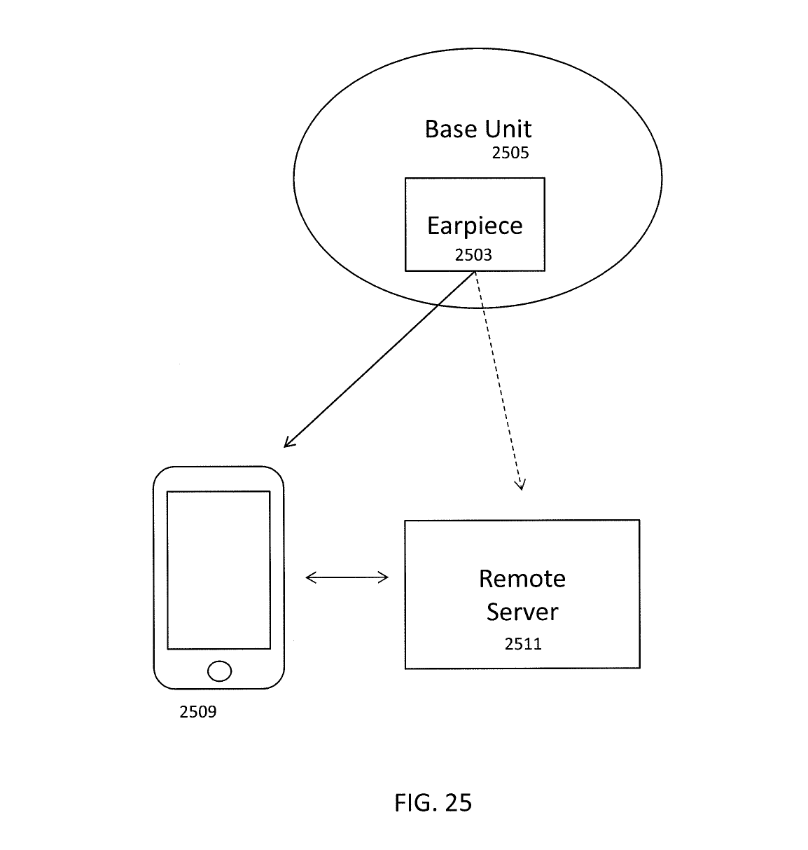

FIGS. 10A and 10B show perspective views of an exemplary base station for receiving and connecting to an earpiece, and FIGS. 21A-21D illustrate another example. The exploded view of FIG. 10A shows the bottom and top of the base station 1001 including a lid 1003 and latch 1005. In general, a docking cradle 1007 (shown in FIG. 10B) may have a plurality of connectors configured to connect with the plurality of electrical contacts on the earpiece. The lid may be secured closed by a latch 1005. In the latch may be configured to hold the lid 1003 closed so that the lid applies pressure against an earpiece when the earpiece is within the docking cradle so that the plurality of connectors maintain electrical contact with the plurality of electrical contacts.

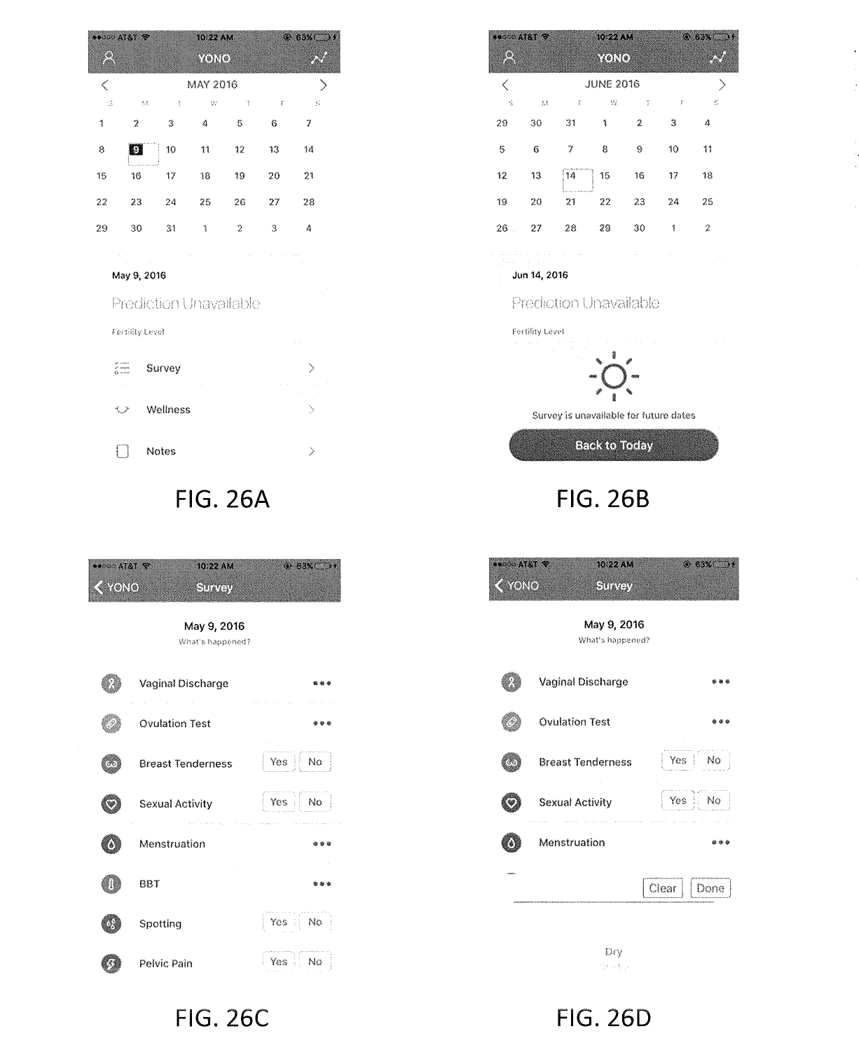

FIGS. 21A-21D illustrate top, top perspective, front and back views, respectively of an exemplary base station. In this example, the base station 2100 is approximately disc-shaped, and includes a lid 2105 with an inwardly-facing projection 2113. As will be shown in FIGS. 22A-22D, this projection, in conjunction with the latch 2107 on the hinged lid 2105 may apply force in the depression region of an earpiece in a manner that helps orient and retain the contact between the electrical connector pins 2122 of the base station and the contacts on the earpiece. The base station shown in FIG. 21D also includes a connector (e.g., USB connector 2109) for connecting to a charger or computer and receiving power and/or transferring data. A hinge 2111 connects the lid to the rest of the base station.

FIGS. 22A-22D illustrate insertion of an earpiece 2201 into an exemplary base station 2200. FIG. 22A shows a base station 2200 with the lid 2205 closed. The lid in this example is hinged and opens as shown in FIG. 22B, exposing the docking cradle having a plurality of connectors (pins 2208) configured to connect with the plurality of electrical contacts on the earpiece 2201 when the earpiece is seated in the docking cradle 2210. In FIG. 22C, an exemplary earpiece 2201 has been inserted into the docking cradle with the top of the earpiece having an indented (concave, depressed) region 2215 exposed on the top. In FIG. 22D, the lid 2205 is again shut, but in this case the lid presses down onto the earpiece 2201 and a latch on the front of the device 2217 releasably latches the lid 2205 over the earpiece, applying a downward force on the earpiece to help maintain contact between the connectors (pins, e.g., pogo pins 2208) and the contacts on the bottom side of the earpiece. In the example shown in FIG. 22B-22D, the projection 2213 on the lid mates with the indented region 2215 on the earpiece, which may help align the earpiece while force is applied to maintain a good electrical contact between the connectors in the docking station and the contacts in the earpiece.

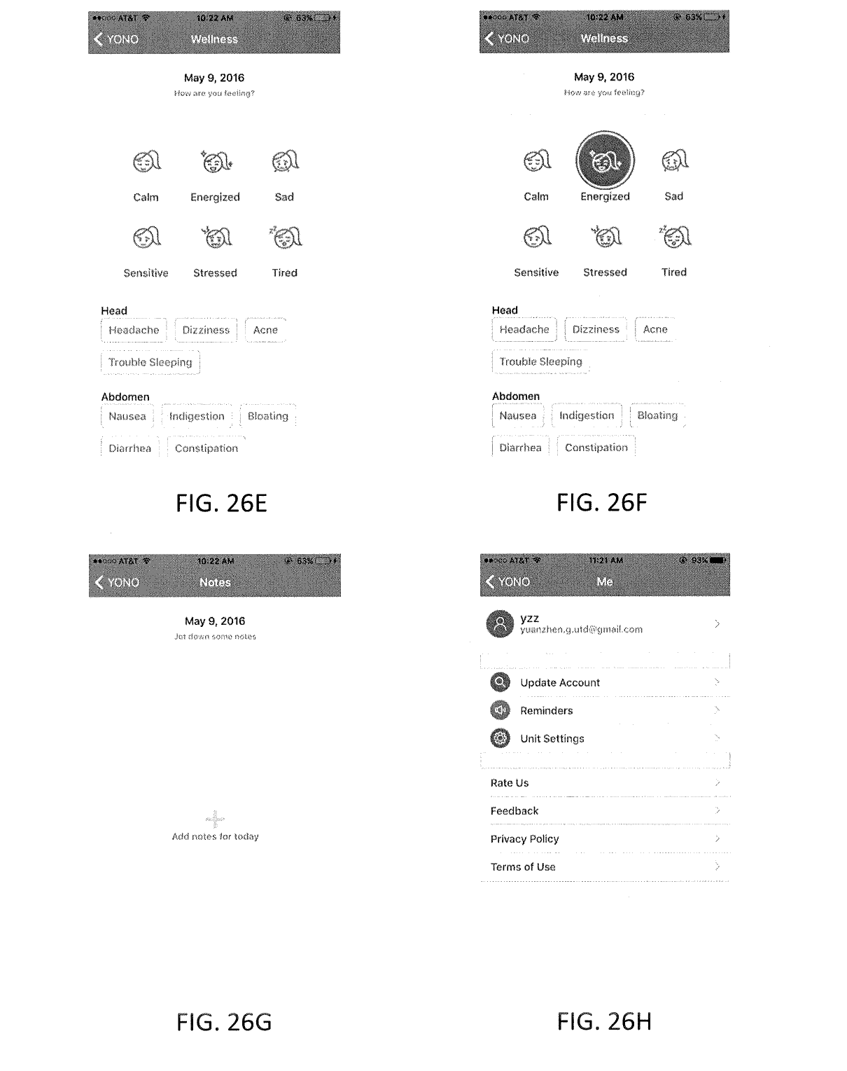

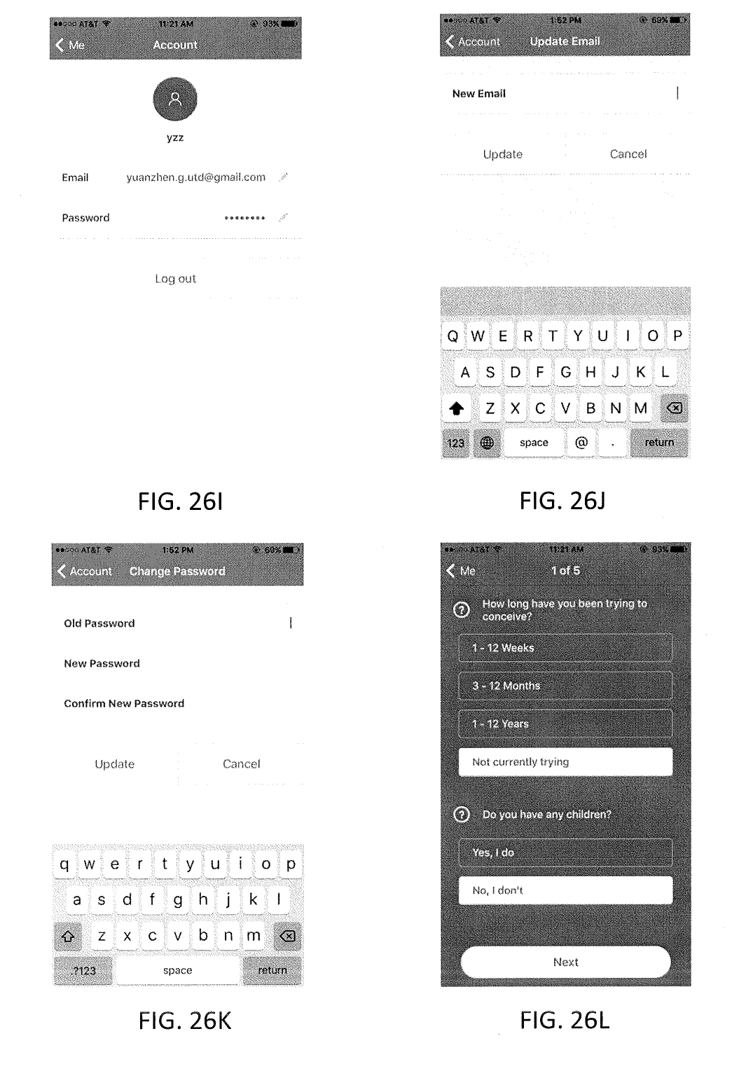

FIGS. 23 and 24 illustrate some exemplary internal structures within the docking station, including a printed circuit board (pcb) 2305 to which power regulator circuitry 2315 is attached. The connector pins 2313 are visible in FIGS. 23 and 24, as well as the cover latch mechanism 2307. In FIG. 24, the lid 2305 is shown covering the cradle region 2325 within which the pins 2313 set. An internal batter 2316 and the pcb substrate 2302 are also visible.

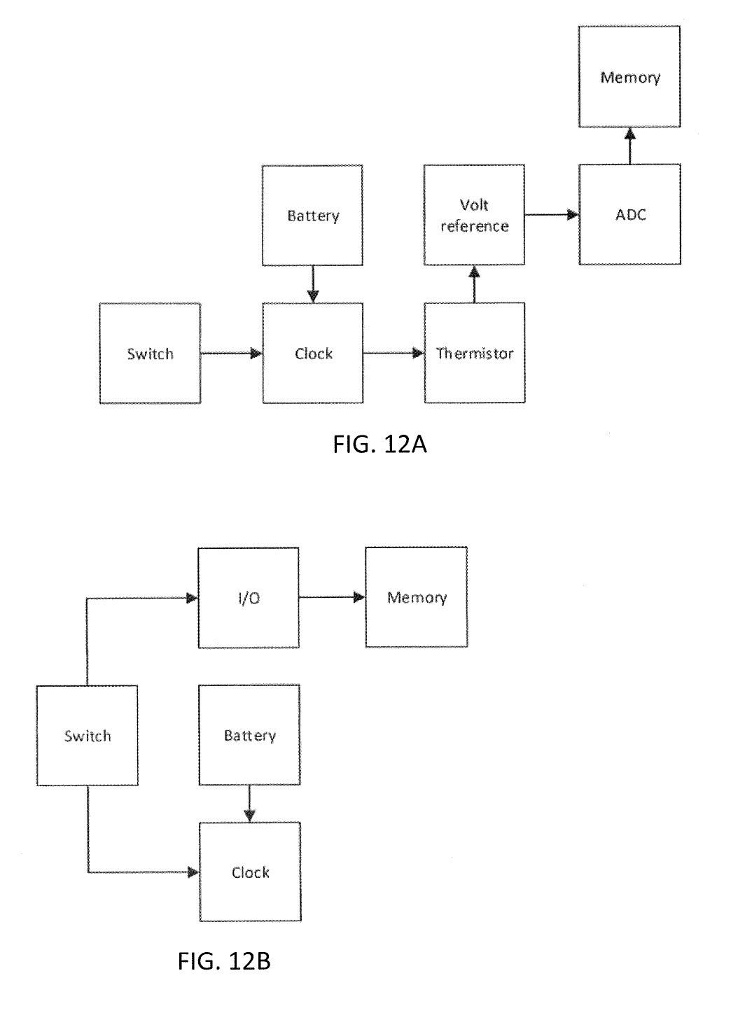

FIGS. 11A-11B show a flowchart illustrating functional steps carried out by the earpiece, a base station and a smartphone.

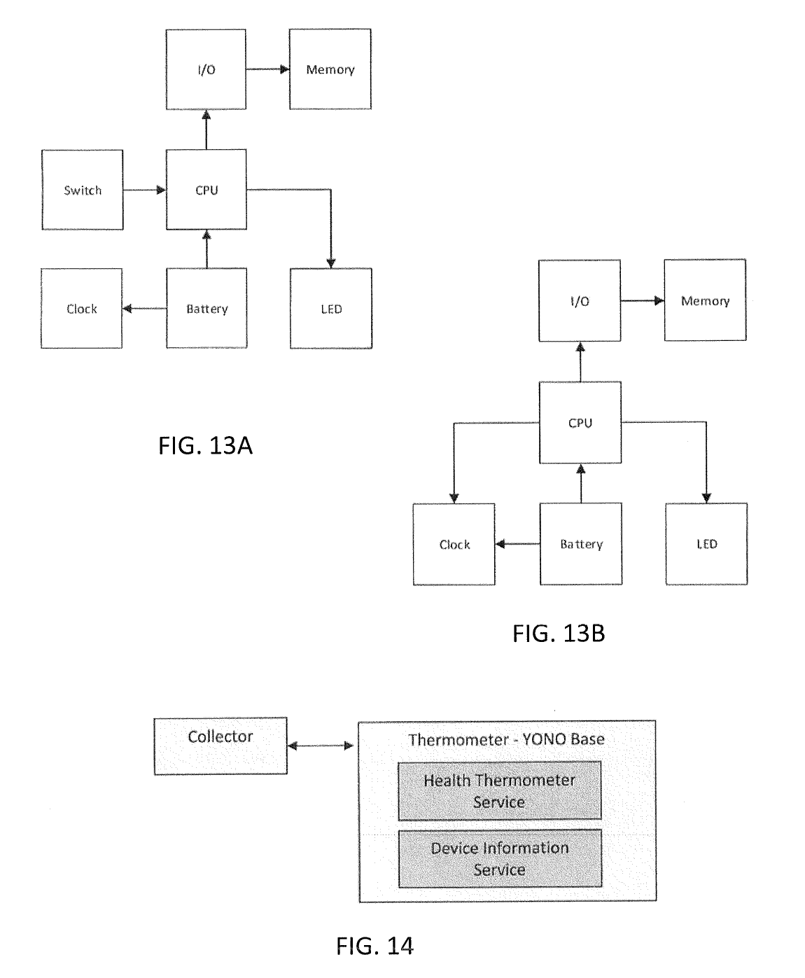

FIGS. 12A and 12B are functional block diagrams of one example of an earpiece applied in sensing and measuring the temperature and in transferring data to the base station. When an earpiece is unplugged from a base station, a switch (e.g., the microcontroller of the earpieces) may be turned to a measurement mode and a clock may start, to activate a temperature measurement procedure periodically according to a preset cycle time of measurements. The temperature sensor (e.g., thermistor) may record the temperature measurement, including the temperature of the air temperature in the close environment enclosed by the earpiece and the ear canal when the earpiece is plugged into the ear. Then an analog to digital converter (ADC) may apply a volt reference to convert the analog signals measured by the earpiece into digital data to store in the memory. After sensing and recording the data, the microprocessor may then switch from the active mode into a sleep mode, to conserve battery power. When the earpiece is plugged onto a base station, the microcontroller may (e.g., after confirming the connection) switch to a docking mode, including a data transfer mode, and the clock may be stopped. The data may be transferred to the base station, or directly to a remote processor (e.g., smartphone) and the memory of the earpiece is emptied and ready for receiving and storing next cycle of temperature measurements.

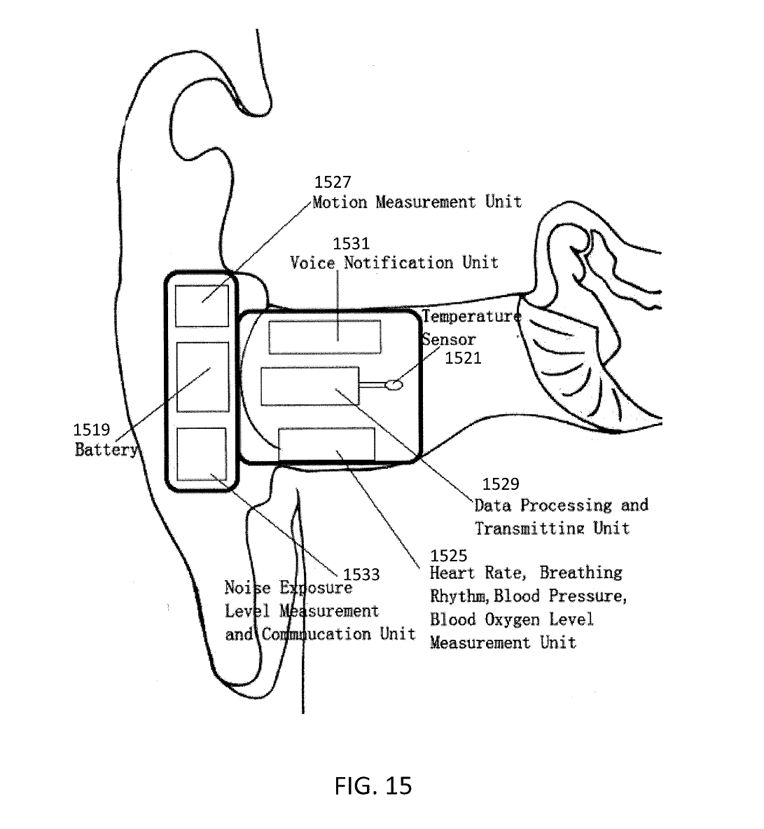

FIGS. 13A and 13B are functional block diagrams of one example of a base station that may retrieve data from the earpiece and to communicate with a smartphone for transferring data to the smartphone. When the earpiece is plugged onto a base station, in some variations the base station may read the data and reads into the memory. Then the base station may start a communication process with a smart phone or a mobile device, the base station transfers the data to the smart phone and the memory of the base station is emptied. As just mentioned, this may be optional, as in some variations the data is not transferred to the base station, but is instead directly passed on by the earpiece when docked into the base station (or in some variations, when worn).

The LED on the base station indicates the on-going process of data transfer. The base station also compares the time to reconstruct the time stamp of the data.

An inner-ear temperature monitor may generally record body temperature while sleeping, e.g., to determine a woman's menstrual cycle. A monitoring device adapted for this purpose may be broken into three primary subsystems: an ear unit (e.g., earpiece), a base station (with attached charging cable), and a smart phone application for displaying the data. An earpiece may include temperature sensing, such as, e.g., a negative temperature coefficient sensor from Measurement Specialties. Part#10K3A1AM, 10K ohm at 25° C., accurate to 0.05° C. from 32° C. to 44° C. A 2.048 volt precision reference may drive a voltage divider that is scaled by a precision operational amplifier to have a 0 to 0.3 volt range over a temperature range of 30° C. to 41° C. The same reference voltage is used for the analog to digital converter in the micro controller.

A microcontroller may be powered by a rechargeable coin cell battery, FDK ML621-TZ1, with a nominal 3V 0.5 mA rating. The earpiece may use an Atmel ATtiny24/44/84 (differing in memory size) to digitize and store the temperature readings (the ATtiny has a 10 bit ADC for 0.01° C. per LSB). The ATtiny may run from its internal oscillator at 128 KHz clock, the slowest frequency supported. It may be configured to come out of its power-saving sleep mode (“Idle Mode”) every 5 minutes to read and store the ear temperature.

An exemplary base station (base unit) may have jumpers that allow operation as either a USB device for programming and data retrieval, or a Bluetooth LE device for data retrieval. Both devices cannot be enables at the same time. The apparatus may include a USB interface. For example, a base unit may have a Micro-USB port for charging base and ear units, programming the ear unit, and retrieving data from the ear unit. Using the USB for programming or data retrieval may include using solder jumpers. USB data may be converted to SPI by a Future Technology Devices FT232H chip. A Bluetooth interface (Laird BL600 FCC approved module based on the Nordic nRF51822 BLE chip) may be used.

The temperature data may be stored in the earpiece in EEPROM using, e.g., 3 bytes per data point: one byte for time stamp, 2 bytes for the 10 bit temperature data. Alternatively, the time stamp may not be used. Total data storage using 3 bytes per reading may allow, e.g., (using the ATtiny 84 having 512 Bytes EEPROM) 171 readings or 14 hours of data. With data compression, the less expensive pin compatible ATtiny24 or ATtiny44 could be used.

Neither the earpiece nor base unit may have a real time clock. Time may be set either by the USB computer of the Bluetooth application and the apparatus may increment a counter to track real time to the nearest minute. Time synchronization can have higher resolution at cost of battery life. When the ear unit is removed from the base station, the software may assume that it is for a data logging run and the base station will start timer. The ear unit will start recording temperature data (e.g., at 5 minute increments), and this will effectively be the ear unit timer. When the ear unit is returned to the base station, the base station may compare its separation timer to the total data logging points stored in the ear unit. The two counts should be within one data recording point of each other. The base station may tag the data as suspect but still allow uploading to the computer or phone app.

When the earpiece is returned to the base station, all the EEPROM logging data may be retrieved from the ear unit and its EEPROM memory erased. The base station LED will indicate data transfer is occurring. In some variations, the retrieved data will may be converted from raw time stamped data to time stamped temperature (e.g., and stored in the Laird 4 Kbytes of EEPROM) or it may be directly transmitted by the earpiece to a handheld (e.g., smartphone) or other receiver. In some variations a full week of data may be stored in the apparatus prior to uploading to, e.g., a phone. The apparatus may use a ringer buffer and overwrite the oldest data as needed. The base unit status LED may indicate when the data buffer is filling up with data that has not been transferred over Bluetooth to the app.

When a base unit is configured as a USB device, the Bluetooth may be disabled and there will be no data storage in the base unit. The base unit will act as a USB pass-through device that allows a PC program to download all the data and clear out the era unit memory to prepare it for another run. Time stamping can also be accomplished by manually entering time data logging started or ended.

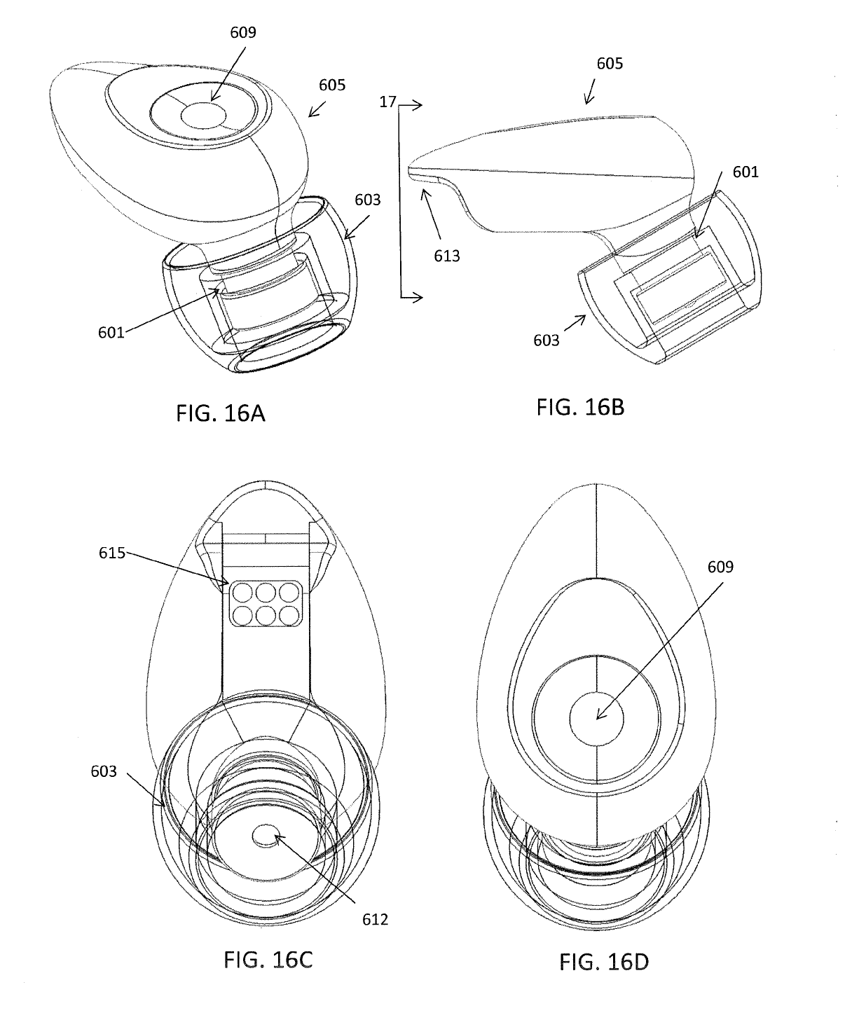

FIG. 14 shows the functional diagram of an example of a Bluetooth software system that uses the Bluetooth Health Thermometer Profile (HTP) and the Bluetooth Health Thermometer Service (HTS) as a guide but does not claim conformance. For example, a connection procedure of un-bonded devices is described below. The earpiece may use the GAP Limited Discoverable Mode when establishing an initial connection. To save power, the earpiece may advertise with an interval of 1 to 3 seconds. The collector may use the Direct Connection Establishment Procedure with a scan interval of 60 to 100 mseconds, and should drop to >2 seconds scan interval after 30 seconds. If a bond is created, the earpiece may write the address of the Collector in the earpiece controller's white list and set the earpiece controller's advertising filter policy to ‘process scan and connection requests only from devices in the White List’. The earpiece may enter a GAP Connectable Mode and start advertising when it has data to send to the Collector. The Collector may execute a GAP connection establishment procedure such that it is scanning for the earpiece using a white list. When the data transfer is complete the earpiece typically terminates the connection.

An earpiece may enter the GAP Undirected Connectable Mode when it has one or more indications or notifications to send to a previously connected Collector. The Collector may use the GAP Direct Connection Establishment Procedure with a scan window/scan interval suitable to its power and connection time requirements. After the earpiece has completed its transfer, it should perform the GAP Terminate Connection procedure after waiting for an idle connection timeout. The earpiece may perform the GAP Terminate Connection procedure if the connection is idle for more than 5 seconds. The earpiece may bond with the Collector and use LE Security Mode 1 and Security Level 2.

In some variations, temperature data may be in ° C. and may include a time stamp field. Alternatively, a time stamp field may not be included but may be calculated later. In one example, an earpiece may include an Atmel ATtiny 84 programmed using the Atmel Studio development environment. It may use its internal 128 Khz Oscillator as a clock source. The ear using communicates with the base station via the SPI bus, and can be reprogrammed by pulling the/RESET line low. The ATtiny may be in Standby Mode when not reading temperatures. This mode allows for the watchdog timer to wake the earpiece up. The earpiece may disable the following functions prior to entering sleep mode: ADC, Analog Comparator, Brown-out Detector, Internal Voltage Reference, and Port Pins. The prescaler of the watchdog timer may be set for a time-out of 8 seconds. The temperature readings are every 10 minutes, thus the ATtiny will read temperatures every 75 wakeups. At every Watchdog Timer wakeup, the ear unit will check to see if there is a connection to the base station.

The ADC may use an external voltage reference (AREF) and read a single ended temperature value on ADC1. Sixteen readings will be combined to produce a single temperature reading that is stored. To minimize CPU work, the sum of multiple readings may be stored, and the conversion to temperature for the base station. Upon disconnection for the base station, the earpiece may start reading temperatures.

In some variations, a separation timer may be used by the base station (e.g., to timestamp the temperature readings).

Sixteen temperature readings may be summed every 10 minutes and stored into EEPROM memory. The readings may span two bytes. In examples using a separation timer, the temperature sum may be read by the base station via the SPI interface.

The earpiece may erase the EEPROM upon the successful transfer of all the data.

In general, the description of the embodiments described herein has been presented for the purpose of illustration and are not intended to be exhaustive or to limit the invention to the precise forms disclosed. Persons skilled in the relevant art can appreciate that many modifications and variations are possible in light of the above disclosure. Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof. Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible computer readable storage medium or any type of media suitable for storing electronic instructions, and coupled to a computer system bus. Furthermore, any computing systems referred to in the specification may include a single processor or may be architectures employing multiple processor designs for increased computing capability.

Embodiments may also relate to a computer data signal embodied in a carrier wave, where the computer data signal includes any embodiment of a computer program product or other data combination described herein. The computer data signal is a product that is presented in a tangible medium or carrier wave and modulated or otherwise encoded in the carrier wave, which is tangible, and transmitted according to any suitable transmission method.

### Additional Sensors

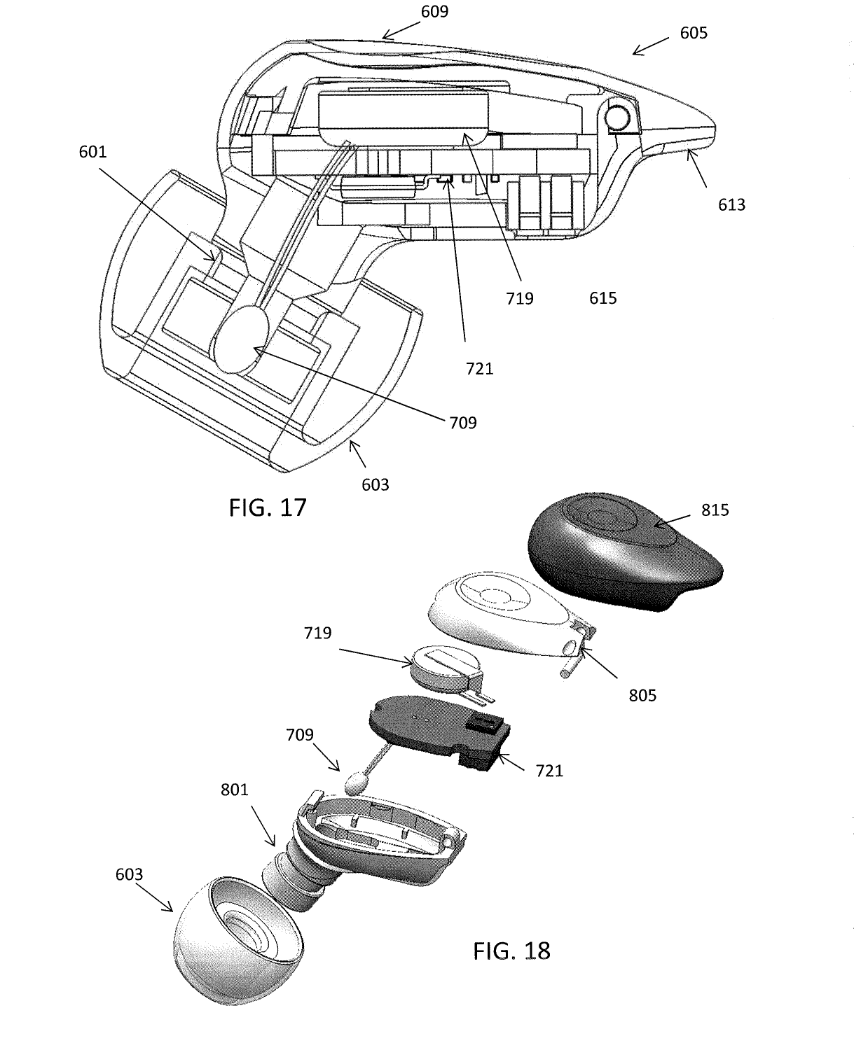

FIG. 15 schematically illustrates another example of a variation of an apparatus as described herein (e.g., an earpiece) having a variety of additional sensors in addition to a temperature sensor.

In this example, the apparatus is similar in shape (having an insertion shaft extending distally from an external seating body), but may include one or more additional sensors, in addition to the temperature sensor such as a thermistor. For example, the apparatus may include sensors to measure heart rate, blood oxygen level, stress level, and noise exposure level. These data may be synced with the temperature data and also transmitted (e.g., to a mobile device or any other external receiving computing device). These devices can be worn in ear for measuring biometrie data such as but not limited to core body temperature, heart rate, breathing rhythm, blood oxygen level, stress level, and noise exposure level. Any one of these additional sensors may be used, or all of them, with any of the earpieces described herein.

The example shown in FIG. 15 also includes a voice notification to announce dramatic changes of the health condition or respond to user's request of current health status. The device could also be used for communication through Bluetooth.

It is both convenient and effective to combine the temperature measurement described above with any of these additional sensors and functionality. In general, measuring biometric data in real-time has its tremendous necessity in medical, industrial and personal use settings. A measuring device worn in ear has its advantages over other locations on the body because there are plenty of blood vessels around the outer ear and in the ear canal. Thus, these earpieces may provide an ideal location for measuring body temperature, heart rate, breathing rhythm, blood oxygen level and etc. Comparing with other wearable devices, a device worn in ear can provide a more stable contact with skin and therefore provide more stable and reliable data.

Any of the earpiece systems described herein which contains multiple biometrie sensors may be combined with an Artificial Intelligent software system. For example a software algorithm may learn the user's living pattern through all the biometrie data collected from the sensors and provide health living advices in real time. The algorithm can also help as assistance in early diagnose of potential health conditions. The device will play a role of private personal health assistant. Although there is no fixed standard to tell if a person is living healthily, an adaptive learning algorithm may be used to analysis the biometrie data collected, and provide user feedback. For example, such a system may let the user know when is the best time to go to sleep if fatigue is detected even though they don't feel sleepy yet, or to awaken, based on a determination of the best time based on body temperature and heart rate data, for example. Thus, in general, any of the apparatuses described herein may include an “alarm clock” function which may be based on absolute time, relative time (e.g., from falling asleep) and/or physiological data collected by the apparatus. For example, a user may be given notice and alarmed when certain vitals of the body is out of the normal range compared to the user's medical records and historical data. This functionality can be used by healthcare personnel to receive an immediate alert about their patient's emergency conditions or by supervisors in factory or construction site to monitor the worker's health status. Users doing exercises can get immediate notice if their heart rate is out of range or their body temperature is too low. Women trying to get pregnant can confirm their pregnancy with the detection of rising heart rate and higher basal temperature data compared with their normal data.

The earpiece apparatuses described herein may also be used to provide data about sleeping cycles. The combination of body temperature, breathing rhythm, and heart rate data of a user in sleep may give an accurate estimation of the user's sleeping cycles. For example, in the first stage of sleeping cycle NREM (non-rapid eye movement), the body temperature usually drops while in the last stage of sleeping cycle REM (rapid eye movement) the body temperature tends to increase a little bit. Heart rate usually slows down in stage 2 of sleeping and reaches the slowest point in stage 3, and then increases again in the final stage before waking up in the morning. Similarly, the change of respiratory patterns can be observed in different sleeping stages during the whole sleeping time.

The earpieces described herein can also be used to monitor workers' health conditions in extreme environmental conditions such as oil field, or construction field which can be relatively hot. According to the industry safety requirement of many governments, employers need to provide sufficient monitoring and preventative equipment to help monitor workers' conditions. The earpieces described herein may combine Bluetooth headset functionality with temperature monitoring to provide warnings when body temperature exceeds a certain level, to minimize heat exhaustion.

In any of the earpieces described herein, the electrical system may include any of the following subsystems: temperature measurement system, heart rate, blood oxygen level and blood pressure measurement system, body movement measurement system, data processing and transmitting system, voice notification system, and communication system.

As illustrated in FIG. 15 and in some variations discussed above, temperature measurement may be achieved by forming a sealed environment in the ear canal and using a thermistor or other type of temperature sensors 1521 to continuously measure the heat radiated by the blood vessel and organs in the inner ear. Heart rate, breathing rhythm, blood oxygen level, blood pressure measurement 1525 may be achieved by using an optical sensor or other types of sensors which consists of several light emitting diodes with different wavelength and a receiver. Body movement measurement system 1527 may use an inertial measurement unit to detect and record the movement of human body, or other types of sensor. Data processing and transmitting systems 1529 may consist of a microprocessor, an ADC, a LDO, and a Bluetooth Low Energy subsystem. Power may be supplied by a battery 1519.

Voice notification 1531, communication 1533, and noise exposure level measurement system 1533 may consist of a audio processor, and a speaker subsystem.

### Application Software

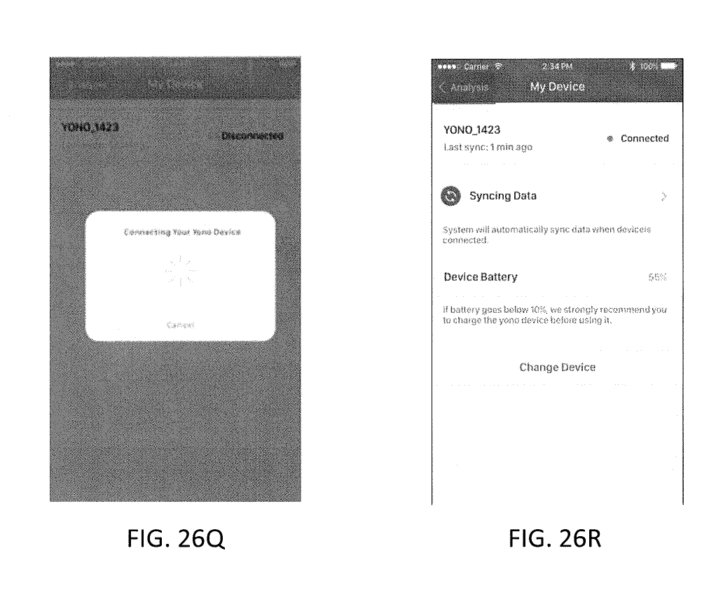

As discussed above, any of the apparatuses described herein may include software, firmware or hardware for receiving and processing the temperature (and any other sensor) data. For example, FIGS. 26A-26R illustrate one example of an application software (“app”) that receives and processes temperature data for use in tracking basal body temperature and predicting (or tracking) ovulation. Such software may be helpful in monitoring fertility for those wishing to conceive (or to avoid conceiving).

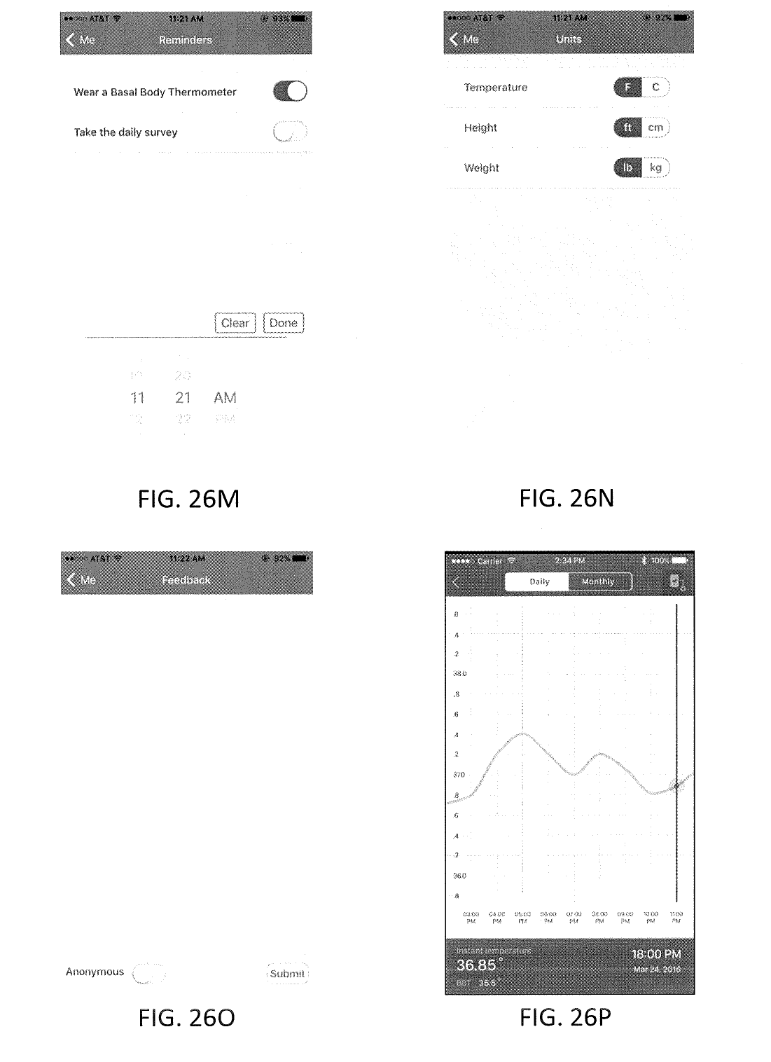

For example, as schematically illustrated in FIG. 25, a system may include an earpiece 2503, such as any of the earpieces described above. The earpiece may dock with a base unit (base station 2505) for charging and (in some variations) transferring data. The earpiece may (e.g., via wireless protocol such as a Bluetooth protocol) transmit directly to a remote processor, including a smartphone 2509. The earpiece may transmit directly to a remote server (e.g., cloud server 2511) or the like, either directly or, alternatively or additionally, via the smartphone 2509 transmitting to the remote server 2511.

In FIG. 25, either or both the smartphone 2509 and the remote server 2511 may include software (e.g. an app or other client software) that causes the processors to receive and manipulate the temperature data. Thus, for example, the application software shown in FIGS. 26A-26R may be executed by a smartphone as part of the systems for determining and/or monitoring body temperature.

In FIG. 26A, a high-level user interface present the body temperature tracking information, including a calendar, a fertility prediction indicator, and links to other pages, including a survey for entering relevant user information about the user's fertility, wellness and any notes to be tracked/recorded. In the background of this screen, the software may communicate and receive the tracking temperature information when the earpiece is docked into the base, as discussed above. In this example, data will be entered into each calendar date sequentially, and this data may be used to predict future fertility, as illustrated in FIG. 26B. In this example, the user may move through the calendar by selecting one or more days, and the screen may show a predicted fertility level (based on body temperature, e.g., basal body temp, from previous entries and/or additional data entered manually by the user). FIGS. 26C-26F illustrate a user manually entering relevant information. Additional notes may also be entered (FIG. 26G) for any particular date. Uses may have an account that is secure (password protected) and includes real-world contact information, as illustrated in FIGS. 26H-25K. Survey information (FIG. 26L-26N) may also be taken, including one-time information such as user height, weight, and preferences (for displaying units, etc.). The application also allows users to send feedback (FIG. 26M).

Temperature data, including basal temperature data, may be presented graphically, as shown in FIG. 26P; graphs may show a single night/day recordings, or multiple nights (one, two week, month, etc.). Non-graphical (e.g., numeric) analyzed data may also be presented. The app may calculate the basal temperature and/or predict fertility (ovulation) or it may communicate the data to a remote server that may calculate this information from the temperature data. The application software may also include control user interfaces showing pairing with the earpiece (FIG. 26Q) and earpiece status (FIG. 26R). In general, the earpiece may be controlled using the apparatus.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for determining body temperature, the system comprising:
an earpiece having:
an insertion shaft extending distally from an external seating body,
a temperature sensor within the insertion shaft,
a microcontroller,
a plurality of electrical contacts electrically connected to the microcontroller, and
a seal around the insertion shaft proximal to the temperature sensor so that the temperature sensor is sealed within the ear canal when the earpiece is worn in a user's ear;
a base station configured to couple with the earpiece, the base station including
a docking cradle having a plurality of connectors configured to connect with the plurality of electrical contacts on the earpiece when the earpiece is seated in the docking cradle; and
a non-transitory computer-readable storage medium storing a set of instructions capable of being executed by a processor, that when executed by the processor causes the processor to:
receive a plurality of temperature data from the earpiece,
determine body temperature from the temperature data, and
display a representation of the body temperature.

2. (canceled)

3. The system of claim 1, wherein the temperature sensor comprises a thermistor.

4. The system of claim 1, wherein the external seating body comprises or is covered in a soft, low-durometer, material.

5. The system of claim 1, wherein the earpiece further comprises an analog-to-digital converter (ADC), and a memory, further wherein the microcontroller is configured to periodically take multiple samples the temperature, average the samples, convert the average from analog to digital and store the average in the memory.

6. The system of claim 1, wherein the plurality of electrical contacts are flush with or recessed into the external seating body.

7. The system of claim 1, further comprising a depression on an upper surface of the external seating body.

8. (canceled)

9. The system of claim 1, further comprising an additional sensor on the earpiece, wherein the additional sensor is selected from one or more of: an optical sensor and an electrical sensor, a noise exposure sensor, a motion detector.

10. The system of claim 1, further comprising one or more additional sensors on the earpiece, wherein the one or more additional sensors are selected from: motion sensor, noise exposer sensor, heart rate sensor, breathing rhythm sensor, blood pressure sensor, and blood oxygen sensor.

11. The system of claim 1, further wherein the base station comprises a lid and latch, wherein the latch is configured to hold the lid closed so that the lid applies pressure against the earpiece when the earpiece is within the docking cradle so that the plurality of connectors maintains electrical contact with the plurality of electrical contacts.

12. (canceled)

13. (canceled)

14. (canceled)

15. The system of claim 1, wherein the non-transitory computer-readable storage medium storing a set of instructions capable of being executed by a processor further causes the processor to request user information comprising one or more of: menstrual data, sexual activity, ovulation, ovulation test data, vaginal discharge.

16. The system of claim 1, wherein the non-transitory computer-readable storage medium storing a set of instructions capable of being executed by a processor further causes the processor to predict ovulation based on basal body temperature.

17. A system for determining body temperature, the system comprising:
an earpiece having:
an insertion shaft extending distally from an external seating body,
a temperature sensor within the insertion shaft,
a microcontroller, and
a plurality of electrical contacts on the external seating body electrically connected to the microcontroller, wherein the electrical contacts are flush with or recessed into the external seating body; and
a base station, the base station including:
a docking cradle having a plurality of connectors configured to connect with the plurality of electrical contacts on the earpiece,
a lid, and
a latch configured to hold the lid closed so that the lid applies pressure against the earpiece when the earpiece is within the docking cradle so that the plurality of connectors maintain electrical contact with the plurality of electrical contacts,
wherein the microcontroller is configured to detect contact between at least one of the electrical contacts and at least one connector for a contact time of greater than 5 seconds before transmitting temperature data from the earpiece.

18. A system for determining body temperature, the system comprising:
an earpiece having:
an insertion shaft extending distally from an external seating body,
a depression on an upper surface of the external seating body,
a temperature sensor within the insertion shaft,
a microcontroller,
a battery, and
a plurality of electrical contacts on the external seating body electrically connected to the microcontroller, wherein the electrical contacts are flush with or recessed into the external seating body; and
a base station, the base station including:
a docking cradle having a plurality of connectors configured to connect with the plurality of electrical contacts on the earpiece,
a lid having a protrusion configured to mate with the depression on the external seating body of the earpiece,
a latch configured to hold the lid closed so that the lid applies pressure against the earpiece when the earpiece is within the docking cradle so that the plurality of connectors maintain electrical contact with the plurality of electrical contacts,
wherein the microcontroller is configured to detect contact between at least one of the electrical contacts and at least one connector for a contact time of greater than 5 seconds before transmitting temperature data from the earpiece.

19. The system of claim 17, wherein the lid of the base station further comprises a protrusion configured to mate with a depression on the external seating body of the earpiece.

20. The system of claim 17, wherein the plurality of electrical contacts comprises plates and the plurality of connectors comprise pogo pin connectors.

21. (canceled)

22. (canceled)

23. The system of claim 17, wherein the temperature sensor comprises a thermistor.

24. The system of claim 17, wherein the external seating body comprises or is covered in a soft, low-durometer, material.

25. The system of claim 17, wherein the earpiece further comprises an analog-to-digital converter (ADC), and a memory, further wherein the microprocessor is configured to periodically take multiple samples of the temperature, average the samples, convert the average from analog to digital and store the average in the memory.

26. The system of claim 17, wherein the plurality of electrical contacts are flush with the external seating body.

27. (canceled)

28. (canceled)

29. The system of claim 17, further comprising one or more additional sensors on the earpiece, wherein the one or more additional sensors are selected from: motion sensor, noise exposer sensor, heart rate sensor, breathing rhythm sensor, blood pressure sensor, and blood oxygen sensor.

30-80. (canceled)
